SEQUENCE LISTING (6 sequences)

SEQ ID NO: 1 - protein, 739 aa, Homo sapiens
TPWSLAWPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 2 - protein, 234 aa, Homo sapiens
TPWSLAWPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKR

SEQ ID NO: 3 - protein, 504 aa, Homo sapiens
KIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 4 - protein, 45 aa, Homo sapiens
KIVLDPSGSMNIYLVLDGSDSIGASNFTGAKKCLVNLIEKVASYG

SEQ ID NO: 5 - protein, 200 aa, Homo sapiens
NIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMI

SEQ ID NO: 6 - protein, 282 aa, Homo sapiens
LCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQD

(19) United States
(12) Patent Application Publication
(10) Pub. No.: US 2010/0015139 A1
(43) Pub. Date: Jan. 21, 2010

(54) METHOD OF INHIBITING COMPLEMENT ACTIVATION WITH FACTOR Ba SPECIFIC ANTIBODIES AND USE THEREOF

(76) Inventor: REKHA BANSAL, Twinsburg, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 12/501,165

(22) Filed: Jul. 10, 2009

Related U.S. Application Data
(60) Provisional application No. 61/079,627, filed on Jul. 10, 2008.

(51) Int. Cl. A61K 39/395

(52) U.S. Cl. 424/133.1; 424/139.1

(57) ABSTRACT

A method of inhibiting the adverse effects of alternative complement pathway activation products in a subject comprising administering to the subject an amount of anti-factor Ba antibody effective to selectively inhibit formation of an alternative complement pathway activation products C3a, C5a, and C5b-9, and activation of neutrophils, monocytes, and platelets.

### RELATED APPLICATION

This application claims priority from U.S. Provisional Application No. 60/968,146, filed Aug. 27, 2007, and U.S. patent application Ser. No. 10/716,929, filed Nov. 19, 2003, the subject matter, which is incorporated herein by reference.

### FIELD OF THE INVENTION

The present invention relates to complement activation. Particularly, the present invention relates to the method for inhibiting complement activation via the alternative pathway. More particularly, the present invention relates to the use of antibodies for factor Ba for inhibiting formation C3bBb or PC3bBb complexes.

### BACKGROUND OF THE INVENTION

The complement system is responsible for initiating and amplifying the inflammatory response to microbial infection and other acute insults. Inappropriate activation of complement has been implicated in pathological situations. For instance, the complement system has been implicated in contributing to the pathogenesis of several acute and chronic conditions, including atherosclerosis, ischemia-reperfusion following acute myocardial infarction, Henoch-Schonlein purpura nephritis, immune complex vasculitis, rheumatoid arthritis, arteritis, aneurysm, stroke, cardiomyopathy, hemorrhagic shock, crush injury, multiple organ failure, hypovolemic shock and intestinal ischemia, transplant rejection, cardiac Surgery, PTCA, spontaneous abortion, neuronal injury, spinal cord injury, myasthenia gravis, Huntington's disease, amyotrophic lateral sclerosis, multiple sclerosis, Guillain Barre syndrome, Parkinson's disease, Alzheimer's disease, acute respiratory distress syndrome, asthma, chronic obstructive pulmonary disease, transfusion-related acute lung injury, acute lung injury, Goodpasture's disease, myocardial infarction, post-cardiopulmonary bypass inflammation, cardiopulmonary bypass, septic shock, transplant rejection, xeno transplantation, bum injury, systemic lupus erythematosus, membranous nephritis, Berger's disease, psoriasis, pemphigoid, dermatomyositis, anti-phospholipid syndrome, inflammatory bowel disease, hemodialysis, leukopheresis, plasmapheresis, heparin-induced extracorporeal membrane oxygenation LDL precipitation, extracorporeal membrane oxygenation, and macular degeneration.

Complement can be activated through three distinct enzymatic cascades, referred to as the “classical”, “Lectin/MBL”, and “alternative” pathways (CP, MBL, and AP respectively). These pathways are shown schematically in FIG. 3. The AP is responsible for 80-95% of total complement activity. Both classical and alternative pathways have the ability to produce C3a and C5a. However, the level of these anaphylatoxins varies depending upon which pathway is active. Lectin pathway is a variation of the classical pathway. Alternative pathway is activated in a number of disease indications. There are three specific proteins Factors B, D, and P that play a major role in the initiation and propagation of the AP. The terminal complex is known as MAC, which is responsible for lysis. Both C3a and C5a are potent anaphylatoxins that are responsible for activating platelets, neutrophils, and monocytes. As a result, inflammatory molecules such as elastase, TNF, IL-1, VEGF, and peroxides are released.

As a result of C3 tick over, C3b is generated. In the schematic, assumption has been made that tick-over of C3 and cleavage of C3 generates the same activated C3b with the released C3a. Activated C3b binds properdin oligomers present in blood to generated (P)n (C3b)n complex. Factor B having higher affinity to properdin bound C3b makes the complex PC3bB, which is then cleaved by factor D to generate PC3bBb. This active convertase cleaves additional C3 to make C3b and release C3a. The same C3 convertase with additional C3b molecules forms C5 convertase. The C5 convertase or C3 convertase cleave C5 to make C5b and C5a. The C5b molecule inserts into the lipid bilayer and forms the nucleus for MAC deposition.

Factor B is composed of two discrete domains Ba (molecular weight, 33kDa) and Bb (molecular weight, 60 kDa). The Ba domain consists of three short consensus repeats known as SCR1, SCR2, and SCR3 (FIG. 1). It has been shown, using mutation analysis and with the use of specific Ba monoclonal antibodies that the factor B functional domain is located in the SCR3 region. Such region was used to produce antibodies that demonstrated clinical benefit in several animal models of diseases. The Bb domain of factor B contains the Von Willowbrand (VWF) domain in addition to the serine protease domain. It is clear from various studies that it is the Ba domain that is important for factor B function, and that inhibition of factor B binding to C3b was required for inhibition of complement activity.

Properdin, a small but important molecule binds C3b to form P-C3b complex and such binding is high affinity. Factor B binds both free C3b and P-C3 to form C3bB and PC3bB complexes. These complexes are cleaved by factor D to form, C3bBb and PC3bBb, both of which possess C3-convertase activity. The resulting convertase can cleave C3, into C3b and C3a. The newly produced C3b fragment, which covalently attaches to the target and then interacts with factors B and D to form the additional alternative pathway C3 convertase molecules.

It is known that the alternative pathway C3-convertase is stabilized by C3b-bound properdin. Since the substrate for the alternative pathway C3-convertase is C3, C3 is therefore both a component and a product of the reaction. As the C3-convertase generates increasing amounts of C3b, an amplification loop is established. Furthermore, the classical pathway can also generate C3b which can bind factor B and thereby engage the alternative pathway even though the trigger is CP mediated. This allows more C3b to deposit on a target leading to enhanced amplification of AP activation. All three, the classical, the lectin, and the alternative pathways converge at C3, which is cleaved by the C3 convertase to form C3b and C3a. C3a is a potent anaphylatoxin and has been implicated in the pathogenesis of a variety of clinical indications. C3a activates neutrophils, monocytes, platelets, mastcells, and T lymphocytes. C3a has been shown to be important for the induction of paw edema in an adjuvant-induced arthritis model.

Addition of newly formed C3b to the existing C3 convertase forms C5 convertase, which cleaves C5 to produce C5b and C5a. C5a similar to C3a is also a potent anaphylatoxin that causes alterations in smooth muscle, in vascular tone, and in vascular permeability. It is also a powerful chemotaxin and an activator of neutrophils, monocytes, platelets, endothelial cells, and T lymphocytes. C5a-mediated cellular activation can significantly amplify inflammatory responses by inducing the release of additional inflammatory mediators, including cytokines, hydrolytic enzymes, arachadonic acid metabolites and reactive oxygen species.

The cleavage of C5 produces C5b and C5a. Anaphylatoxin C5a is released and C5b inserts itself into the lipid bilayer and acts as a nucleus for C6, C7, C8, and C9 deposition to form the C5b-9 complex at the surface of the target cell. C5b-9 is also known as the membrane attack complex (MAC). There is now strong evidence that MAC may play an important role in inflammation in addition to its role as a lytic pore-forming complex. In addition to the proven role of C3a, C5a in platelet activation, C5b-9 is also known to mediate activation of platelets. Thus, there is significant evidence suggesting C3a, C5a, and MAC involvement in activation of platelets. Regardless of the method of platelet activation, activated platelets express CD62P also called P-selectin. P-selectin also mediates platelet-monocyte conjugation, and such binding triggers the release of tissue factor from monocytes. One result of such conjugate formation is the removal of platelets from the circulation, a phenomenon that can contribute to the development of thrombocytopenia.

While complement activation provides a valuable first-line defense against potential pathogens, the activities of complement that promote a protective inflammatory response can also represent a potential threat to the host. For example, C3a and C5a anaphylatoxins recruit and activate neutrophils, monocytes and platelets to the pathological site. These activated cells are indiscriminate in their release of destructive enzymes and may cause organ damage. Currently, there are no approved drugs exist that can inhibit the damages caused by the inappropriate activation of the complement pathway. Based upon the available clinical data, it appears that in most acute injury settings, complement activation is mediated predominantly via the alternative pathway. Therefore, developing suitable methods that inhibit only this pathway without completely obviating the immune defense capabilities would be highly desirable. This would leave the classical pathway intact to handle immune complex processing and to aid in host defense against infection.

### SUMMARY OF THE INVENTION

In one aspect, the present invention provides a method of inhibiting the adverse effects of factor B-dependent complement activation in a living subject. The method includes the step of administering to a subject in need thereof, an amount of an anti-factor Ba antibody effective to inhibit factor B-dependent complement activation. In this context, the phrase “factor B-dependent complement activation” refers to alternative pathway complement activation. In another aspect of the invention, the anti-factor Ba antibody also inhibits the amplification loop of the classical complement pathway.

In another aspect, the present invention provides the use of the anti-factor Ba antibody or antigen binding fragment thereof in a variety of acute and chronic clinical conditions.

The methods, compositions and medicaments of the invention are useful for inhibiting the adverse effects of factor-B-dependent complement activation in vivo in mammalian subjects, including humans suffering from an acute or chronic pathological condition or injury as further described herein. Such conditions and injuries include without limitation factor-B mediated complement activation in associated autoimmune disorders and/or inflammatory conditions.

In another aspect of the invention, methods are provided for inhibiting factor-Ba-dependent complement activation in a subject suffering from ischemia reperfusion injuries by treating a subject experiencing myocardial infarction, ischemic reperfusion, cardiopulmonary bypass including without limitation, after aortic aneurysm repair, , vascular reanastomosis in connection with, for example, organ transplants (e.g., heart, lung, liver, kidney) and/or extremity/digit replantation, stroke, hemodynamic resuscitation, and atherosclerosis following shock and/or surgical procedures, with a therapeutically effective amount of a factor-B antibody in a pharmaceutical carrier.

In another aspect of the invention, methods are provided for inhibiting factor-B-dependent complement activation in a subject suffering from vascular condition, including without limitation cardiovascular conditions, cerebrovascular conditions, peripheral (e.g., musculoskeletal) vascular conditions, renovascular conditions, mesenteric/enteric vascular, and revascularization to transplants and/or replants, by treating such patient with a therapeutically effective amount of a factor-B antibody. Such conditions include without limitation the treatment of: vasculitis, including Henoch-Schonlein purpura nephritis, SLE-associated vasculitis, vasculitis associated with rheumatoid arthritis (also called malignant rheumatoid arthritis), immune complex vasculitis, and Takayasu's disease; dilated cardiomyopathy; diabetic angiopathy; Kawasaki's disease (arteritis); venous gas embolus (VGE); and/or restenosis following stent placement, rotational atherectomy and/or percutaneous transluminal coronary angioplasty (PTCA).

In another aspect of the invention, methods are provided for inhibiting factor-B-dependent complement activation in a subject suffering from inflammatory gastrointestinal disorders, including but not limited to pancreatitis, diverticulitis and bowel disorders including Crohn's disease, ulcerative colitis, and irritable bowel syndrome.

In another aspect of the invention, methods are provided for inhibiting factor-B-dependent complement activation in a subject suffering from a pulmonary condition including but not limited to acute respiratory distress syndrome, transfusion-related acute lung injury, ischemia/reperfusion acute lung injury, chronic obstructive pulmonary disease, asthma, Wegener's granulomatosis, antiglomerular basement membrane disease (Goodpasture's disease), meconium aspiration syndrome, bronchiolitis obliterans syndrome, idiopathic pulmonary fibrosis, acute lung injury secondary to bum, non-cardiogenic pulmonary edema, transfusion-related respiratory depression, and emphysema.

In another aspect of the invention, methods are provided for inhibiting factor-B-dependent complement activation in a subject that has undergone, is undergoing or will undergo an extracorporeal reperfusion procedure, including but not limited to hemodialysis, plasmapheresis, leukopheresis, extracorporeal membrane oxygenation (ECMO), heparin-induced extracorporeal membrane oxygenation LDL precipitation (HELP) and CPB.

In another aspect of the invention, methods are provided for inhibiting factor-B-dependent complement activation in a subject suffering from a musculoskeletal condition, including but not limited to osteoarthritis, rheumatoid arthritis, juvenile rheumatoid arthritis, gout, neuropathic arthropathy, psoriatic arthritis, ankylosing spondylitis or other spondyloarthropathies and crystalline arthropathies, muscular dystrophy or SLE.

In another aspect of the invention, methods are provided for inhibiting factor-B-dependent complement activation in a subject suffering from renal conditions including but not limited to mesangioproliferative glomerulonephritis, membranous glomerulonephritis, membranoproliferative glomerulonephritis (mesangiocapillary glomerulonephritis), acute postinfectious glomerulonephritis (poststreptococcal glomerulonephritis), cryoglobulinemic glomerulonephritis, lupus nephritis, Henoch-Schonlein purpura nephritis or IgA nephropathy.

In another aspect of the invention, methods are provided for inhibiting factor-B-dependent complement activation in a subject suffering from psoriasis, autoimmune bullous dermatoses, eosinophilic spongiosis, bullous pemphigoid, epidermolysis bullosa acquisita and herpes gestationis and other skin disorders, or from a thermal or chemical burn injury involving capillary leakage.

In another aspect of the invention, methods are provided for inhibiting factor-B-dependent complement activation in tissue transplant, including but not limited to allotransplantation or xenotransplantation of whole organs (e.g., kidney, heart, liver, pancreas, lung, cornea, etc.) or grafts (e.g., valves, tendons, bone marrow, etc.).

In another aspect of the invention, methods are provided for inhibiting factor-B-dependent complement activation in a subject suffering from a CNS disorder or injury or a PNS disorder or injury, including but not limited to multiple sclerosis (MS), myasthenia gravis (MG), Huntington's disease (HD), amyotrophic lateral sclerosis (ALS), Guillain Barre syndrome, reperfusion following stroke, degenerative discs, cerebral trauma, Parkinson's disease (PD), Alzheimer's disease (AD), Miller-Fisher syndrome, cerebral trauma and/or hemorrhage, demyelination and meningitis.

In another aspect of the invention, methods are provided for inhibiting factor-B-dependent complement activation in a subject suffering from a blood disorder including but not limited to sepsis or a condition resulting from sepsis including without limitation severe sepsis, septic shock, acute respiratory distress syndrome resulting from sepsis, and systemic inflammatory response syndrome. Related methods are provided for the treatment of other blood disorders, including hemorrhagic shock, hemolytic anemia, autoimmune thrombotic thrombocytopenic purpura (TTP), hemolytic uremic syndrome (HUS) or other marrow/blood destructive conditions.

In another aspect of the invention, methods are provided for inhibiting factor-B-dependent complement activation in a subject suffering from a urogenital condition including but not limited to painful bladder disease, sensory bladder disease, chronic abacterial cystitis and interstitial cystitis, male and female infertility, placental dysfunction and miscarriage and pre-eclampsia.

In another aspect of the invention, methods are provided for inhibiting factor-B-dependent complement activation in a subject suffering from nonobese diabetes (Type-1 diabetes or Insulin dependent diabetes mellitus) or from angiopathy, neuropathy or retinopathy complications of Type-1 or Type-2 (adult onset) diabetes.

In another aspect of the invention, methods are provided for inhibiting factor-B-dependent complement activation in a subject being treated with chemotherapeutics and/or radiation therapy, including without limitation for the treatment of cancerous conditions, by administering a factor-B inhibitor to such a patient perichemotherapeutically or periradiation therapy, i.e., before and/or during and/or after the administration of chemotherapeutic(s) and/or radiation therapy. Perichemotherapeutic or periradiation therapy administration of factor-B inhibitors may be useful for reducing the side-effects of chemotherapeutic or radiation therapy. In a still further aspect of the invention, methods are provided for the treatment of malignancies by administering a factor-B antibody in a pharmaceutically acceptable carrier to a patient suffering from a malignancy.

In another aspect of the invention methods are provided for inhibiting factor-B-dependent complement activation in a subject suffering from an endocrine disorder, by administering a therapeutically effective amount of a anti-factor Ba antibody in a pharmaceutical carrier to such a subject. Conditions subject to treatment in accordance with the present invention include, by way of nonlimiting example, stress, Hashimoto's thyroiditis anxiety and other potential hormonal disorders involving regulated release of prolactin, growth or insulin-like growth factor, and adrenocorticotropin from the pituitary.

In another aspect of the invention methods are provided for inhibiting factor-B-dependent complement activation in a subject suffering from age-related macular degeneration or other complement mediated ophthalmologic condition by administering a therapeutically effective amount of an anti-factor Ba antibody in a mammal.

### BRIEF DESCRIPTION OF THE DRAWINGS

FIG. 1 illustrates a schematic of factor B.

FIG. 2 is a schematic illustration of the mechanism of interaction of a prior art monoclonal antibody with Factor B and its fragment Ba.

FIG. 3 illustrates factor B Binds Properdin Bound C3b with High Affinity: This figure compares the binding affinity of Factor B binding to C3b and properdin bound C3b. Various concentrations of factor B were added to C3b coated and Properdin bound C3b coated plates. Properdin bound C3b enhances the binding of factor B to C3b by -20 fold. In this assay, the Kd of binding to C3b is ˜19.4 nM and the binding to properdin bound C3b is 1.11 nM.

FIG. 4 illustrates factor B Does Not Bind C3b Isoforms in the Presence/Absence of Properdin: This figure demonstrates factor B binding to the various isoforms iC3b, C3c, and C3dg. This assay was performed in the presence and absence of properdin. Various concentration of factor B was added to the plates coated with the isoforms. The results from this figure demonstrate that factor B does not bind to the isoforms of C3b.

FIG. 5 illustrates human Factor B does not Bind Properdin: This figure demonstrates that factor B does not bind to properdin. To understand the mechanism of how the convertase is stabilized, we evaluated whether there are molecular interactions between factor B and properdin. Various concentrations of properdin was added to substrate bound factor B. The results indicate that properdin does not bind to factor B.

FIG. 6 illustrates binding of Anti-Ba to Ba Protein: Factor B consists of two unique domains, the Ba domain, and the Bb domain. Reports indicate that the Ba is responsible for initial C3bB formation upon which factor D cleaves factor B, releasing the Ba component and leaving the Bb component attached to C3b. This assay evaluated the binding affinities of a Ba specific antibody and Bb specific antibody. The results of this assay show that the antibodies bind to distinctly different regions of factor B and there is no overlap of the antibody binding domains. Anti-Ba binds to Ba protein with a Kd of 9.65 nM whereas Anti-Bb does not bind to Ba.

FIG. 7 illustrates Anti-Ba Inhibits Factor B Binding to Properdin Bound C3b: The Ba domain of factor B is responsible for initial association of factor B to C3b to form the C3bB complex. This complex stability is enhanced by properdin binding. Anti-Ba binds to the Ba domain of factor B and prevents factor B association with C3b thus inhibiting the formation of the PC3bB complex. The assay demonstrates that Anti-Ba inhibits B binding to properdin bound C3b with an IC50 of 234 nM.

FIG. 8 illustrates Anti-Ba Inhibits Factor B Binding to C3b: This assay is similar to the assay performed for FIG. 26. The only difference is that C3b is not bound to properdin. The IC50 of this assay is 556 nM.

FIG. 9 illustrates Anti-Ba Inhibits AP Dependent C3-Converatase Formation in Normal Human Serum (Inhibition of C5b-9 Formation): This assay evaluated the ability of Anti-Ba to inhibit alternative pathway activation of normal human serum via LPS stimulation. C5b-9 is the terminal component of the complement cascade and is responsible for many deleterious effects when inappropriately activated. Various concentrations of Anti-Ba was added to normal human serum and incubated on LPS coated plates. LPS stimulates normal human serum and activates the alternative pathway. C3-convertase is formed and leads to the formation of C5b-9 via the cleavage of C5. This assay demonstrates that Anti-ba is able to inhibit convertase formation and thus C5b-9 formation but not as effectively as NMOO1. Anti-Ba was able to inhibit this activity with an IC50 of -527 nM.

FIG. 10 illustrates Anti-Ba Inhibits AP Dependent Hemolysis: The hemolysis assay is another measure of C5b-9 formation. This assay was performed in comparison to an Anti-Bb antibody at similar concentrations. The assay demonstrates that Anti-Ba can inhibit hemolysis but not as effectively as Anti-Bb (FIG. 30). Anti-Ba is able to inhibit at 250 μg/ml but loses its effect at 125 μg/ml.

FIG. 11 illustrates Anti-Ba Inhibits C3a Formation during Extracorporeal Circulation of Whole Human Blood: The tubing loop model is an ex vivo extracorporeal circulation model that causes alternative pathway activation of the blood by contact with the artificial surfaces of the tubing loop. The alternative pathway forms the C3-convertase, which cleaves C3 into C3a and C3b. C3a is a potent anaphylatoxin able to activate many inflammatory cells. Various concentrations of Anti-Ba were incubated with whole human blood and rotated through the tubing loop. After rotation, the plasma was extracted and evaluated for C3a product. Anti-Ba was able to inhibit C3a production dose dependently.

FIG. 12 illustrates Anti-Ba Inhibits Elastase Formation during Extracorporeal Circulation of Whole Human Blood: Elastase release is considered a marker of neutrophil activation. Neutrophils are activated by the anaphylatoxins C3a and C5a. To measure the effect of Anti-Ba on elastase formation, tubing loop plasma samples were evaluated in a sandwich ELISA assay. As shown, Anti-Ba dose dependently inhibited neutrophil elastase formation.

FIG. 13 illustrates Anti-Ba Inhibits TNF-alpha Formation during Extracorporeal Circulation of Whole Human Blood: TNF-Alpha release is considered a marker of monocyte activation. Monocytes are activated by the anaphylatoxins C3a and C5a. To measure the effect of Anti-Ba on TNF-alpha formation, tubing loop plasma samples were evaluated in a sandwich ELISA assay. As shown, Anti-Ba dose dependently inhibited TNF-alpha formation.

FIG. 14 illustrates Anti-Ba Inhibits Neutrophil Activation during Extracorporeal Circulation of Whole Human Blood: Alternative complement pathway activation produces the anaphylatoxins C3a and C5a as a result of C3-converatase cleavage of C3 and C5 into C3a and C5a. These anaphylatoxins are peptides that bind to their respective receptors on neutrophils and monocytes and cause the release of inflammatory chemicals and proteins. The most prominent of these released components are TNF-Alpha, Neutrophil Elastase, and IL-1Beta. Anti-Ba is able to inhibit alternative complement activation thus prevent the formation of the anaphylatoxins that cause the activation of neutrophils, monocytes, and platelets. Using flow cytometry and the appropriate cellular activation markers, Anti-Ba effects on cellular activation was evaluated. This figure demonstrates dose dependent inhibition of Anti-Ba on neutrophil activation.

### DETAILED DESCRIPTION

Unless specifically addressed herein, all terms used have the same meaning as would be understood by those of skilled in the art of the present invention. The following definitions will provide clarity with respect to the terms used in the specification and claims to describe the present invention.

The term “alternative pathway” mainly refers to complement activation which is triggered by artificial surfaces such as; lipopolysaccharide (LPS) from Gram negative outer membranes, and rabbit erythrocytes, zymosan from fungal and yeast cell walls, as well as from many pure polysaccharides, viruses, bacteria, animal tumor cells, parasites and damaged cells, and which are thought to activate C3 into a conformationally active molecules with the ability to bind factor Ba.

The term “lectin pathway” refers to complement activation that involves specific binding of mannan-binding lectin (MBL) and the ficolins.

The term “classical pathway” refers to complement activation which is triggered by antigen-antibody complexes and requires C1Q for activation and alternative pathway amplification loop for its propagation, where the contribution from the amplification loop is nearly 85-90%.

As used herein, the term “alternative complement inhibitory agent” refers to an antibody molecule that binds to or directly interacts with factor B and effectively inhibits factor B dependent complement activation. Anti-factor B antibodies useful in the method of the invention may reduce factor B-dependent complement activation by greater than 20%, such as greater than 50%, such as greater than 90%. In one embodiment, the anti-factor B antibody reduces factor B-dependent complement activation by greater than 90%.

As used herein, the term “antibody” encompasses antibodies and antibody fragments thereof, derived from any antibody-producing mammal (e.g., mouse, rat, rabbit, and primate including human), that specifically bind to factor Ba polypeptides or portions thereof. Exemplary antibodies include monoclonal, polyclonal, recombinant antibodies; multispecific antibodies (e.g., bispecific antibodies); humanized antibodies; murine antibodies; chimeric, mouse-human, mouse-primate, primate-human monoclonal antibodies; and anti-idiotype antibodies, and may be any intact molecule or fragment thereof.

As used herein, the term “antibody fragment” refers to a portion derived from a full-length anti-factor B antibody, generally including the antigen binding and variable region thereof. Other antibodies include diabodies, linear antibodies, single-chain antibody molecules and multispecific antibodies formed from antibody fragments. Examples of antibody fragments include Fab, Fab′, F(ab)2, F(ab′)2 and Fv fragments, or scFv fragments.

As used herein, a “single-chain Fv” or “scFv” antibody fragment comprises the VH and VL domains of an antibody, wherein these domains are present in a single polypeptide chain.

As used herein, a “chimeric antibody” contains the variable domains and complementarity-determining regions derived from a non-human species (e.g., rodent) antibody, and the remainder of the antibody molecule is derived from a human antibody.

As used herein, a “humanized antibody” is a chimeric antibody that consists of a sequence that conforms to specific complementarity-determining regions derived from non-human immunoglobulin that is grafted into a human antibody framework. Humanized antibodies are recombinant proteins in which only the antibody complementarity-determining regions are of non-human origin.

As used herein, the “membrane attack complex” (“MAC”) refers to the terminal complex of the complement pathways where C5b an integral component of the MAC inserts itself into the lipid bilar for the assembly of C5b-9 complex.

As used herein, “a subject” includes all mammals, including without limitation humans, non-human primates, dogs, cats, horses, sheep, goats, cows, rabbits, pigs and rodents.

The term “polypeptide” is used herein as a generic term to refer to native protein, fragments, or analogs of a polypeptide sequence. Hence, native protein, fragments, and analogs are species of the polypeptide genus.

The term “polypeptide fragment” as used herein refers to a polypeptide that has an amino-terminal and/or carboxyl-terminal deletion, but where the remaining amino acid sequence is identical to the corresponding positions in the naturally occurring sequence deduced, for example, from a full-length properdin sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long.

The term “monoclonal” refers to an antibody that binds to a sequence of amino acid and has a single specific epitope on its target antigen. For example, anti-factor Ba antibody is a monoclonal antibody that is specific to the Ba domain of factor B. Because the antibody is monoclonal, it would recognize a domain/motif that contains the sequence contained in Ba (SEQ ID NO: 2).

The term “polyclonal” refers to an antibody that recognizes multiple epitope sites on a single antigen. For example, a polyclonal antibody against Ba indicates that the antibody will bind several sites of the Ba.

The term “epitope” includes any protein determinant capable of specific binding to an immunoglobulin. Epitope determinants usually consist of chemically active surface groupings of molecules such as amino acids or sugar side chains and usually have specific three-dimensional structural characteristics, as well as specific charge characteristics.

The terms “oligomer” and “polymer” are used interchangeable. The terms “oligomer” and “polymer” refer to the association of more than one monomer of a specific protein, peptide, or peptide fragments. The terms “oligomer” and “polymer” in this invention specifically relates to the ability of properdin protein monomers to form protein complexes with it or with other proteins.

The terms “patient,” “mammalian host,” and the like are used interchangeably herein, and refer to mammals, including human and veterinary subjects.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease.

The term “a disease or disorder associated with the alternative complement pathway,” as used herein, refers to a disease or disorder caused, directly or indirectly, by activation of the alternative complement pathway, a disease or disorder that is mediated, directly or indirectly, by one or more components of the alternative complement pathway, or a product generated by the alternative complement pathway. The term also refers to a disease or disorder that is exacerbated by one or more components of the alternative complement pathway, or a product generated by the alternative complement pathway.

The term “knockout” refers to the technique in which a specific gene(s) are removed from a target animal. This technique is usually applied to rodents in which the gene of interest is removed via homologous recombination of an empty vector with the native animal chromosome. The technique works by swapping the animal's chromosome containing the gene with the empty vector containing a marker or random DNA sequences. This method results in an animal that is deficient of the gene of interest. The present invention would utilize this technique to generate antibodies against an antigen that is removed from the animal's genome to enhance generation of antibodies.

It is to be understood that this invention is not limited to particular embodiments described, as such may, of course, vary. It is also to be understood that the terminology used herein is for describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are encompassed within the invention, subject to any specifically excluded limit in the stated range. Where the stated range includes one or both of the limits, ranges excluding either or both of those included limits are also included in the invention.

The present invention relates to a method of a method of inhibiting alternative complement pathway activation in a mammal, inhibiting the adverse effects of alternative complement pathway activation products, and/or treating alternative pathway activation mediated by disease-related or pathological conditions. The method includes administering to a mammal an anti-factor Ba antibody that inhibits the formation of C3bBb and PC3bBb, prevents the release of Ba from the PC3bB complex, inhibits the formation of C3a, C3b, C5a, C5b-9, and sC5b-9, inhibits the formation of Ba, inhibits the formation of Bb, prevents the formation of TNF alpha and neutrophil elstaste, and inhibitis the activation of neutrophils, monocytes, and platelets in clinical conditions where the disease pathology is complement-mediated.

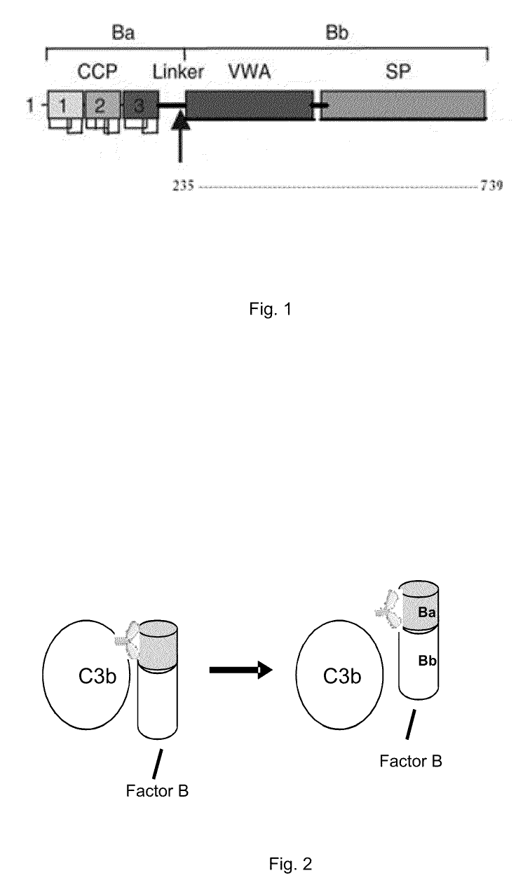

Factor B is a 90 kDa protein that can have an amino acid sequence of SEQ ID NO: 1. Factor B consists of three domains (FIG. 1): a three-module complement control protein (CCP1, CCP2, and CCP3), a von Willebrand factor A domain (e.g., SEQ ID NO: 5), and a C-terminal serine protease (SP) domain (e.g., SEQ ID NO: 6) that adopts a default inactive (zymogen) conformation. The interaction between factor B and surface-bound C3b triggers a conformational change in factor B that ultimately creates the “C3 convertase” (PC3bBb) of the alternative complement pathway. The activation of the alternative pathway of complement (AP) hinges on a Magnesium ion-enhanced interaction between factor B and C3b. Upon binding, factor B is rendered susceptible to proteolytic cleavage by factor D, forming fragments Ba (30 kDa) (e.g., SEQ ID NO: 2) and Bb (60 kDa) (e.g., SEQ ID NO: 3). Bb, in association with C3b, comprises the AP C3 convertase. This complex has serine protease activity and functions to cleave native C3 into C3a and C3b.

Factor B is activated through an assembly process: it binds surface-bound C3b after which it is cleaved by factor D into fragments Ba (residues 26-234; SEQ ID NO: 2) and Bb (residues 235-739 SEQ ID NO: 3). Fragment Ba dissociates from the complex, leaving behind the alternative pathway C3 convertase complex C3b-Bb, which cleaves C3 into C3a and C3b. This protease complex is intrinsically instable. Once dissociated from the complex, Bb cannot reassociate with C3b. The proenzyme factor B consists of three N-terminal complement control protein (CCP: 1, 2, and 3) domains, connected by a 45-residue linker (SEQ ID NO: 4) to a VWA domain and a C-terminal serine protease (SP) domain, which carries the catalytic center triad (ASP-HIS-SER). The VWA and SP domains form fragment Bb, and CCP1 through CCP3 and the linker form fragment Ba. Binding of factor B to C3b depends on elements in fragment Ba and the Mg2+-dependent metal ion-dependent adhesion site (MIDAS) motif in the VWA domain of fragment Bb.

In accordance with an aspect of the invention, the anti-factor Ba antibody is directed to or specifically binds to the Ba domain of factor B. The anti-factor Ba antibody of this invention binds the Ba fragments, does not bind Bb fragment, can inhibit the factor B binding to C3b, inhibits C3b production, inhibits C3a, C5a, C5b-9 formation and inhibits lysis of rabbit erythrocytes. As illustrated in FIG. 2, the anti-Ba antibody does not bind the Bb segment and is involved only in Factor B binding to C3b and cleavage of factor B by factor D to form factor Ba.

The anti-factor Ba monoclonal antibodies of the present invention prevent formation of of C3b. If C3b formation is completely prevented properdin will float alone without any C3b attached. Properdin does not bind C3 or the isoforms of C3b.

In another aspect of the invention, the anti-factor Ba antibody can be specific to inhibiting the alternative pathway and not inhibit the classical pathway, which is generally required for host defense against infection. The anti-factor Ba antibody can also inhibit C3a, C5a, and C5b-9 and cellular activation. Both C3a and C5a are potent anaphylatoxins that are generated during the AP activation. Regardless of the trigger/initiator of the AP, if C3a, C5a and C5b-9 are formed in excessive amounts above control levels, damage to cellular systems occurs. Neutrophils bear the C5a receptor and therefore respond to compounds that prevent C5a production or antibodies that neutralize the C5a or the receptor antagonists that prevent receptor attack by C5a. Similarly, platelets have the C3a receptors and therefore agents that prevent/neutralize C3a activity would prevent platelet activation. Monocytes have C3a receptors and upon activation release “TNF” and “IL-1” which have been implicated in inflammatory diseases such as arthritis. Secretory components such as neutrophil elastase, TNF, and IL-1 have been defined as the markers of inflammation. In addition activated neutrophils, monocytes and platelets orchestrate the inflammatory responses by forming leukocyte-platelet conjugates. In a number of clinical diseases, all these cell types are known.

Anti-factor Ba antibodies of the present invention can include murine human anti-factor Ba monoclonal antibodies, and compositions comprising the antibodies. The anti-factor Ba antibody can be produced by an antibody-producing hybridoma. Chimeric, de-immunized, single chain, truncated, fully human and humanized versions of the anti-factor Ba antibody can be generated by those skilled in the art. Human/humanized/chimerized anti-factor Ba antibody avoids problems associated with rodent antibodies, i.e., adverse reactions in humans, such as hypersensitivity reactions, including urticaria, dyspnea, hypotension, anaphylaxis, and the like.

An example of an anti-factor Ba antibody in accordance with the present invention that specifically binds to the Ba region and, as described in the Examples, that can inhibit the complement activation is commercially available from Quidel, San Diego, Calif. under the trade name A225. The Quidel antibody is listed as being a murine anti-Factor Ba antibody. It was discovered that this antibody specifically binds to factor Ba and shows no reactivity to the peptide Bb. This antibody is also described in U.S. patent application Ser. No. 10/716,929, which is incorporated by reference in its entirety, as specifically inhibiting the alternative complement pathway.

Another aspect of the invention relates to antibodies that bind to the same epitope on Ba as the anti-factor Ba antibody described in the Examples (i.e., the Quidel anti-factor B antibody). Such antibodies can be identified based on their ability to cross-compete with the anti-factor Ba antibody of the Examples in standard Ba binding assays. The ability of a test antibody to inhibit the binding of the anti-factor Ba antibody of the Examples to Ba demonstrates that the test antibody can compete with the anti-factor Ba antibody of the Examples for binding to Ba and thus binds to the same epitope on Ba as the anti-factor Ba antibody of the present invention. In an aspect of the invention, the antibody that binds to the same epitope on Ba as the anti-factor Ba antibody of the Examples is a human monoclonal antibody.

In yet another aspect, an antibody of the invention can comprise heavy and light chain variable regions comprising amino acid sequences that are homologous to the amino acid sequences of the preferred antibodies described herein, and wherein the antibodies retain the desired functional properties of the anti-factor Ba antibodies of the invention. For example, the invention provides an isolated monoclonal antibody, or antigen binding portion thereof, comprising a heavy chain variable region and a light chain variable region, wherein: (a) the heavy chain variable region comprises an amino acid sequence that is at least 80% homologous to the amino acid sequence of heavy chain of the anti-factor Ba antibody (b) the light chain variable region comprises an amino acid sequence that is at least 80% homologous to the amino acid sequence of the anti-factor Ba antibody; and (c) the antibody specifically binds to the Ba region of factor B.

In various aspects, the antibody can be, for example, a human antibody, a humanized antibody or a chimeric antibody. In other aspects, the VH and/or VL amino acid sequences may be 85%, 90%, 95%, 96%, 97%, 98% or 99% homologous to the sequences set forth above. An antibody having VH and VL regions having high (i.e., 80% or greater) homology to the VH and VL regions of the sequences set forth above, can be obtained by mutagenesis (e.g., site-directed or PCR-mediated mutagenesis) of nucleic acid molecules the heavy and light chain of the anti-factor Ba antibody, followed by testing of the encoded altered antibody for retained function (i.e., the functions set forth in (c) and (d) above) using the functional assays described herein.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

In certain aspects, an antibody of the invention can include a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein. In a more specific example, the heavy chain variable region CDR2 sequence comprises the amino acid sequence of heavy chain of the anti-factor Ba antibody, or conservative modifications thereof; and the light chain variable region CDR2 sequence comprises the amino acid sequence light chain of the anti-factor Ba antibody, or conservative modifications thereof.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the functions set forth in (c) through (j) above) using the functional assays described herein.

An antibody of the invention further can be prepared using an antibody having one or more of the VH and/or VL sequences disclosed herein as starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties. Thus, such antibodies contain the VH and VL CDR sequences of the anti-factor Ba antibody may contain different framework sequences from these antibodies.

Another type of variable region modification is to mutate amino acid residues within the VH and/or VK CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Conservative modifications (as discussed above) are introduced. The mutations may be amino acid substitutions, additions or deletions, but are preferably substitutions. Moreover, typically no more than one, two, three, four or five residues within a CDR region are altered.

Anti-factor Ba monoclonal antibodies can be prepared by standard methods well known in the art. For example, rodents (e.g. mice, rats, hamsters, and guinea pigs) can be immunized either with factor B, or factor Ba purified from human plasma or urine or with recombinant factor B or its fragments expressed by either eukaryotic or prokaryotic systems. Antibodies specific to factor Ba can be identified by selecting those that do not bind the Ba. The antibody product of the present invention does not bind the Ba region of the protein. Other animals can also be used for immunization, e.g. non-human primates, transgenic mice expressing human immunoglobulins, and severe combined immunodeficient mice transplanted with human B-lymphocytes. Hybridoma can be generated by conventional procedures well known in the art by fusing B lymphocytes from the immunized animals with myeloma cells (e.g. Sp2/0 and NSO). In addition, anti-factor Ba antibodies can be generated by screening of recombinant single-chain Fv or Fab libraries from human B lymphocytes in phage-display systems. The specificity of the MoAbs to human factor Ba can be tested by enzyme linked immunosorbent assay (ELISA).

In the antibody molecule, there are four chains. The amino-terminal portion of each chain includes a variable region of 100 to 110 amino acids responsible for antigen recognition. The carboxyl-terminal portion of each chain defines a constant region primarily responsible for effector function. Thus, an intact antibody has two binding sites. Except in bifunctional or bispecific antibodies, the two binding sites are the same. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv).

Human antibodies can be prepared that avoid certain of the problems associated with antibodies that possess murine or rat variable and/or constant regions. An important practical application of such a strategy is the “humanization” of the mouse humoral immune system. Introduction of human immunoglobulin (Ig) loci into mice in which the endogenous IgG genes have been inactivated offers the opportunity to study the mechanisms underlying programmed expression and assembly of antibodies as well as their role in B-cell development. Furthermore, such a strategy could provide an ideal source for production of fully human monoclonal antibodies (MoAbs)—an important milestone towards fulfilling the promise of antibody therapy in human disease. Fully human antibodies are expected to minimize the immunogenic and allergic responses intrinsic to mouse or mouse-derivatized MoAbs and thus to increase the efficacy and safety of the administered antibodies. The use of fully human antibodies can be expected to provide a substantial advantage in the treatment of chronic and recurring human diseases, such as inflammation, autoimmunity, and cancer, which require repeated antibody administrations.

One approach towards this goal is to engineer mouse strains deficient in mouse antibody production with large fragments of the human IgG loci in anticipation that such mice would produce a large repertoire of human antibodies in the absence of mouse antibodies. Large human IgG fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human MoAbs with the desired specificity could be readily produced and selected.

While chimeric antibodies have a human constant region and a murine variable region, it is expected that certain human anti-chimeric antibody (HACA) responses will be observed, particularly in chronic or multi-dose utilizations of the antibody. Thus, it would be desirable to provide fully human antibodies against Ba in order to vitiate concerns and/or effects of HAMA or HACA response.

### Humanization and Display Technologies

As was discussed above in connection with human antibody generation, there are advantages to producing antibodies with reduced immunogenicity. To a degree, this can be accomplished in connection with techniques of humanization and display techniques using appropriate libraries. It will be appreciated that murine antibodies or antibodies from other species can be humanized or primatized using techniques well known in the art.

Antibody fragments, such as Fv, F(ab′)₂ and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Further, human antibodies or antibodies from other species can be generated through display-type technologies, including, without limitation, phage display, retroviral display, ribosomal display, and other techniques, using techniques well known in the art and the resulting molecules can be subjected to additional maturation, such as affinity maturation, as such techniques are well known in the art.

### Additional Criteria for Antibody Therapeutics

As discussed herein, the function of a subject anti-Ba antibody appears important to at least a portion of its mode of operation. By function, we mean, by way of example, the activity of the anti-Ba antibody in inhibiting the alternative complement pathway, e.g., a subject anti-Ba antibody exhibits one or more of the following properties: (a) inhibiting the formation of C3b/PC3b bound Bb; (b) inhibiting the factor D cleavage of factor B, (c) inhibiting the formation of C3a, C3b, C5a, C5b-9, and sC5b-9, (d) inhibiting the formation of C3bBb and PC3bBb, (e) inhibiting the formation of Bb, (f) inhibiting the formation of Ba, and (g) inhibiting the activation of neutrophils, monocytes, and platelets

### Design and Generation of Other Therapeutics

Other therapeutic modalities beyond antibody moieties can be facilitated. Such modalities include, without limitation, advanced antibody therapeutics, such as bispecific antibodies, immunotoxins, and radiolabeled therapeutics, generation of peptide therapeutics, gene therapies, particularly intrabodies, antisense therapeutics, and small molecules. In connection with the generation of advanced antibody therapeutics, where complement fixation is a desirable attribute, it may be possible to sidestep the dependence on complement for cell killing using bispecifics, immunotoxins, or radiolabels, for example.

Bispecific antibodies can be generated that comprise (i) two antibodies one with a specificity to Ba and another to a second molecule that are conjugated together, (ii) a single antibody that has one chain specific to Ba and a second chain specific to a second molecule, or (iii) a single chain antibody that has specificity to Ba and the other molecule. Such bispecific antibodies can be generated using techniques that are well known for example, in connection with (i) and (ii) and in connection with (iii).

In connection with the generation of therapeutic peptides, through the utilization of structural information related to Ba and antibodies thereto, such as the antibodies of the invention (as discussed below in connection with small molecules) or screening of peptide libraries, therapeutic peptides can be generated that are directed against Ba.

Assuming that the Ba molecule (or a form, such as a splice variant or alternate form) is functionally active in a disease process, it will also be possible to design gene and antisense therapeutics thereto through conventional techniques. Such modalities can be utilized for modulating the function of Ba.

### Therapeutic Administration and Formulations

It will be appreciated that the therapeutic entities in accordance with the invention will be administered with suitable carriers, excipients, and other agents that are incorporated into formulations to provide improved transfer, delivery, tolerance, and the like. These formulations include, for example, powders, pastes, ointments, jellies, waxes, oils, lipids, lipid (cationic or anionic) containing vesicles (such as Lipofectin), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, if the active ingredient in the formulation is not inactivated by the formulation and the formulation is physiologically compatible and tolerable with the route of administration.

### Preparation of Antibodies

Antibodies in accordance with the invention are prepared in mouse using standard methods well know in the art. The monoclonal antibody of the present invention will be converted into a humanized version for therapeutic use. The antibody can be made by contract or in house into humanized, fully human, chimeric, recombinant for therapeutic use. The hybridoma cell lines discussed herein are readily generated by those of ordinary skill in the art, given the guidance provided herein. Each of the antibodies produced by the subject cell lines are those that do not generate an adverse response. Adverse response is defined as unwanted responses which means in this invention an antibody type that can inhibit alternative pathway complement activation.

Antibodies in accordance with the present invention can be expressed in cell lines other than hybridoma cell lines. Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. Cell lines of particular preference are selected through determining which cell lines have high expression levels and produce antibodies with constitutive Ba binding properties.

The results of the present invention indicate that antibodies can be made more efficacious than currently available antibodies against Ba and therefore will be efficacious in treating disorders associated with and/or mediated by the alternative complement pathway.

### Therapeutic Uses

The complement system provides an early acting mechanism to initiate and amplify the inflammatory response to microbial infection and other acute insults. The complement system has been implicated as contributing to the pathogenesis of several acute and chronic disease conditions, including: myocardial infarction, paroxysmal nocturnal hemoglobinuria, hemolytic anemia, spinal cord injuries, revascularization following stroke, ARDS, reperfusion injury, septic shock, capillary leakage following thermal burns, postcardiopulmonary bypass inflammation, transplant rejection, rheumatoid arthritis, multiple sclerosis, myasthenia gravis, and Alzheimer's disease to mention a few. Pathological conditions where complement byproducts are found elevated would fall under the regime of complement mediated inflammatory conditions In these conditions, complement may not be the cause but is certainly one of many factors involved in pathogenesis. Nevertheless, control of complement activation may be a major mechanism to prevent and/or downregulate destruction associated with inflammation.

The complement system can be activated via three distinct pathways: the classical pathway, the lectin pathway, and the alternative pathway. The classical pathway is triggered by antibody bound to a foreign particle (possibly an antigen). In contrast, both the lectin and the alternative pathways are part of the innate immune system.

Regardless of the trigger agent, all three pathways engage in the amplification loop of the alternative pathway. The C3 convertases of all three pathways cleave C3 into C3b which can participate with factor B in forming additional alternative pathway C3 convertase (C3bBb). Cleavage of C3 by C3 convertase produces C3a and cleavage of C5 by C5 convertases generates C5b and C5a. Both C3a and C3b are major anaphylatoxins responsible for pathological outcomes. C3a activates monocytes and platelets, C5a is the most potent anaphylatoxin, inducing alterations in smooth muscle and vascular tone, as well as vascular permeability. It is also a powerful chemotaxin and activator of both neutrophils and monocytes. C5a-mediated cellular activation can significantly amplify inflammatory responses by inducing the release of multiple additional inflammatory mediators, including cytokines, hydrolytic enzymes, arachidonic acid metabolites and reactive oxygen species. C5b moves on to produce C5b-9, also known as the membrane attack complex (MAC). There is now strong evidence that sublytic MAC deposition may play an important role in inflammation in addition to its role as a lytic pore-forming complex.

### Role of Alternative Pathway in Classical Pathway Trigerred Activation of Complement:

As discussed above, C3b is the integral part of the C5 convertase of the classical pathway, and of C3/C5 convertases of the alternative pathway. The C3 convertase of the classical pathway (C4bC2a) cleaves C3 into C3b and C3a. The C3b molecule generated as a result of classical pathway activation can bind properdin and factor B of the alternative pathway to generate the alternative pathway C3a and C5 convertases. As a result, the amplification loop takes off and starts generating additional C3b molecule thus providing amplification and perpetuation of the classical pathway via this amplification loop. The alternative pathway, in addition to its widely accepted role as an independent pathway for complement activation, also provides an amplification loop for complement activation that is initially triggered via the classical and lectin pathways.

Role of the alternative pathway in various diseases: Despite its essential role in immune defense, the complement system contributes to tissue damage in many clinical conditions. Thus, there is a pressing need to develop therapeutically effective complement inhibitors to prevent these adverse effects. The preferred protein component to target in the development of therapeutic agents, is factor Ba binding to C3b to inhibit the alternative pathway complement contribution to the pathology of diseases selected from the group consisting of, myocardial infarction, ischemia/reperfusion injury, stroke, acute respiratory distress syndrome (ARDS), burn injury, cardiopulmonary bypass inflammation, extracoporeal circulation, radiographic contrast media induced allergic response, transplant rejection, multiple sclerosis, myasthenia gravis, pancreatitis, rheumatoid arthritis, Alzheimer's disease, asthma, thermal injury, anaphylactic shock, bowel inflammation, urticaria, angioedema, vasculitis, Sjogren's syndrome, lupus erythromatosus, and membranous nephritis, dermatomyositis, vascular stenosis and restenosis.

### Role of Alternative Complement Pathway in Extracorporeal Circulation (ECC) Cardiopulmonary Bypass:

These procedures refer to the surgical process during which blood is diverted from a patient's circulatory system to an extracorporeal circulation systems or ECC. It is an important medical technology that is used in cardiac surgeries, valve replacements, cardiac injuries, hemodialysis, plasmapheresis, plateletpheresis, leukophereses, extracorporeal membrane oxygenation (ECMO), heparin-induced extracorporeal LDL precipitation (HELP), and cardiopulmonary bypass (CPB). ECC is for coronary artery bypass grafting (CABG) utilized for many types of open heart surgeries, including congenital heart defects, heart valve disease, or other heart defects. In all these conditions, blood is exposed to foreign surfaces that alters normal hemostasis and often leads to activation of neutrophils, monocytes, and platelets. For example, in cardiopulmonary bypass (CPB) surgery, blood is routed through the Heart-and-Lung machine where patient's blood comes in contact with surfaces of the long tubings, oxygenator, and filters. Ultimately a systemic inflammatory reaction sets in that can cause significant inflammatory responses. The CPB-triggered inflammatory reaction can result in post-surgical complications, generally termed “postperfusion syndrome.” Included under which are cognitive deficits, respiratory failure, bleeding disorders, renal dysfunction and, in the most severe cases, multiple organ failure. Patients who have an exaggerated inflammatory response to CPB tend to bleed more, require more respiratory support, demonstrate greater capillary leakage via weight gain, and display a decline in independent functioning relative to normal responders. Thus, it appears that the magnitude of the inflammatory response to CPB adversely influences clinical outcomes. Coronary bypass surgery with CPB (on pump) leads to profound activation of complement compared to surgery without CPB (off pump). A number of studies evaluated complement activation byproducts such as C3a, C5a, and sC5b-9 and nearly all such studies suggested significant elevation in concentration of such products. Both C3a and C5a are potent stimulators of neutrophils, monocytes, and platelets. MAC also has been shown to activate neutrophil and platelets during ECC. Activation of these cells results in release of proinflammatory cytokines (IL-1, IL-6, IL-8, TNF alpha), Complement activation and release of tumour necrosis factor alpha, interleukin-2, interleukin-6 and soluble tumour necrosis factor and interleukin-2 receptors during and after cardiopulmonary bypass in children. C5a has been shown to upregulate expression of the adhesion molecules, CD11b and CD 18 of Mac-1 in polymorphonuclear cells (PMNs) and to induce degranulation of PMNs to release proinflammatory enzymes. C5b-9 induces the expression of the adhesion molecule, P-selectin (CD62P), on platelets, whereas both C5a and C5b-9 induce surface expression of P-selectin on endothelial cells. These adhesion molecules are involved in cell to cell interaction among leukocytes, platelets and endothelial cells. The expression of adhesion molecules on activated endothelial cells is responsible for sequestration of activated leukocytes at sites of inflammation. These cells then mediate tissue inflammation and injury. It is the actions of these complement activation products on neutrophils, monocytes, platelets and other circulatory cells that likely lead to the various problems that arise after CPB.

The complement activation byproducts, C3a, C5a, and sC5b-9 (MAC), are known to activate all three cell types via C3a and C5a receptors or by direct insertion of MAC into the cellular bilayer. Both, C3a and C5a receptors have been found on neutrophils, monocytes, and T lymphocytes. Activation of proinflammatory cells causes elevated levels of elastase, peroxides, and a whole range of cytokines including TNF alpha and IL-1 beta that contribute to the inflammatory response. Activated platelets can become dysfunctional depending upon the potency of stimulus. Complement-mediated platelet dysfunction can result in both excessive thrombosis and excessive bleeding as platelets first become activated and then become spent and non-functional, and are removed from the circulation. Such bleeding complications are costly and can require blood transfusions to save the patients.

ECC clearly causes complement activation, as demonstrated by the sequence of inflammatory events that are known to occur when cardiac patients go for bypass procedures. There is a huge literature available on post perfusion syndrome also known as post CPB syndrome. In such inflammatory conditions, elevated levels of C3a, C5a, and MAC have been found in plasma. In addition, activated neutrophils, monocytes, and platelets are found in blood following CPB. Inflammatory byproducts of activated inflammatory cells are also found in the plasma, these include; TNF, elastase, and peroxides. The established tubing loop model of CPB was originally established by Gong et al and later modified by Gupta-Bansal to evaluate complement and cellular activation markers of inflammation following the rotation of blood in a loop of tubing. The tubing loop model of CPB uses method that are similar to those used in the clinic during bypass where blood is diluted with plasmalyte and subjected to the ECC model of CBP. As a result of blood contact coming in contact with the artificial surface of the tubing, both complement and cellular activation of neutrophils, monocytes and platelets occurs.

C3a activates monocytes, lymphocytes, promotes arthritis, causes the release of IL-6 and IL-8, and is known to cause cognitive impairment. C3a has also been implicated in urticaria, rhinitis and asthma. New findings indicate that the C3a/C5a receptors are widely expressed in neurons and astrocytes and may modulate neuronal function. C5a is another anaphylatoxin that is involved in induction of arthritis Essential role for the C5a receptor in regulating the effector phase of synovial infiltration and joint destruction in experimental arthritis., brain inflammation, Expression of the complement C3a and C5a receptors after permanent focal ischemia: An alternative interpretation and asthma, Complement factors C3a and C5a are increased in bronchoalveolar lavage fluid after segmental allergen provocation in subjects with asthma. Both C3a and C5a cause platelet aggregation, Induction of platelet aggregation by the complement-derived peptides C3a and C5a. The anaphylatoxins, C3a and C5a, which are releases with activation of complement components, trigger mast cell degranulation, which releases histamine and other mediators of inflammation that results in early events that set the stage for inflammation, such as smooth muscle contraction, increased vascular permeability, leukocyte activation, and other inflammatory phenomena. C5a also functions as a chemotactic peptide that serves to attract pro-inflammatory granulocytes to the site of complement activation. In addition, direct activation of endothelial cells by C5a induces the release of the coagulation-inhibiting glycoprotein heparan sulfate.

The cleavage product of C5, C5b, combines with C6, C7, and C8 to form the C5b-9 complex at the surface of the target cell. After a sufficient number of MACs insert into target cell membranes, the openings (MAC pores) these MACs create mediate rapid osmotic lysis of the target cells—an exaplme is tissue injury and other diseases where erythrocyte lysis is the hallmark of disease pathology.

Effect of CPB/ECC on Neutrophil Pathophysiology: Anaphylatoxins are known to activate neutrophils by upregulating the expression of CD11b during bypass. As a result, neutrophils adhere to the endothelium of blood vessels and start cytotoxic events. In CPB, elevation in CD11b levels have been reported CD11b on neutrohils is also associated with respiratory burst and myosite injury. Activated neutrophils, upon degranulation, release elastase, lactoferin, and myeloperoxidase, substances which clearly play roles in acute lung injury in patients undergoing bypass. Elastase secretion has been shown to help in neutrophil transmigration through the endothelial barrier. In contrast to neutrophils, where the predominant inflammatory response is initiated by the C5a and C5b-9 molecules of complement, monocyte activation and activation of CD11b is primarily regulated by C3a in CPB. Platelets bear both C3a and C5a receptors that respond to individual stimuli in guinea pig blood.

In an extracorporeal setting, anti-factor P, C5, and D monoclonal antibodies have been used to downregulate platelet activation in the tubing loop model of CPB. Such monoclonal antibodies and a small molecule complement inhibitor also have been used in a baboon model of CPB with similar results. Both models, with and without the animal attached, demonstrate activation of neutrophils, monocytes, and platelets as a result of complement activation.

Platelet dysfunction can also occur when platelets come in contact with the non-biological surfaces of the ECC associated with CPB. Additional mechanisms, for example, mechanical trauma due to shear stress, surface adherence, and turbulence within the extracorporeal oxygenator, may cause fragmentation of platelet membranes. Activated complement components, however, are arguably the most important factors contributing to the platelet dysfunction that are observed in associated with ECC. Within minutes after initiating CPB, bleeding time is prolonged significantly and platelet aggregation is impaired due to platelet dysfunction. These changes in bleeding time are independent of platelet count and worsen as CPB progresses. Activated platelets express CD62P as an activation marker. This protein is a specific integral membrane protein also known as GMP-140. Several reports suggest that the expression of P-selectin, an activation marker of monocytes and neutrophils, is directly associated with platelet dysfunction, and occurs during ECC. Effects of ECC on platelets are particularly significant because platelets can only be activated once, i.e., activation of platelets decreases the number of functional platelets available when platelet functions are subsequently required. The importance of the effects of ECC on platelets is demonstrated by the finding that the impaired hemostasis observed after cardiac operations is mainly attributable to platelet dysfunction. The activation marker, CD62P, is known to mediate the binding of platelets to leukocytes, thus causing the formation of platelet-neutrophil and platelet-monocyte conjugates. One result of such conjugate formation is the removal of platelets from the circulation, a phenomenon that can contribute to the development of thrombocytopenia.

CPB effects on neutrophil and monocytes activation have been described in relation to the expression of the activation marker for these cell, CD11b. Upregulation in the activation of neutrophils and monocytes is particularly relevant to CPB-induced injury since expression of CD11b/CD18 is responsible for leukocyte adherence to and penetration (diapedesis) through the endothelium via binding to the intercellular adhesion molecules ICAM-1 and ICAM-3 on “activated” endothelium. In addition, increased CD11b expression on leukocytes has been linked to complications associated with hemodialysis. Thus, based on these findings, CD11b/CD18 may contribute to ECC associated medical problems. Platelets also form conjugates with monocytes. Platelet-monocyte conjugates have been reported to occur during hemodialysis and have been identified as markers of myocardial infarction. In addition, there is strong evidence that suggests monocyte activation by C3a induces monocytes to produce and release of proinflammatory mediators such a as interleukin 1beta and TNF-alpha. Thus, collectively, the elevated levels of C3a, C5a, and MAC are responsible for cellular activation which in turn causes tissue and cellular damages seen in post perfusion syndrome complications of CPB or ECC. Activated monocytes and neutrophils also accumulate in the pulmonary vessels and vascular beds, as has been demonstrated by serial biopsies of lung tissue before and after CPB and contribute to postoperative dysfunction of the lungs. Liver, brain and pancreas, also suffer such damage, which can result in postoperative dysfunction of these organs.

Tubing Loop Model of Cardiopulmonary Bypass: The tubing loop model of CPB has been widely used to assess the effect of complement inhibitory drugs in the extra corporeal model. The model mimics many features of the CPB machine, but without the attached patient. Complement and cellular activation products are elevated in samples after whole blood samples are rotated in the tubing loop, these include significantly elevated levels of C3a, C5a, sC5b-9, neutrophil elastase, and TNF. Cellular analysis of blood aliquots after rotation in the tubing loop also demonstrates elevated levels of CD11b positive neutrophils and monocytes, and CD62P positive platelets. Further, analyses of serum samples indicate elevated levels of neutrophil elastase and TNF.

One aspect of the invention is thus directed to the prevention or treatment of extracorporeal exposure-triggered inflammatory reaction by treating a subject undergoing an extracorporeal circulation procedure with a therapeutically effective amount of a factor B antibody in a pharmaceutical carrier, including patients undergoing hemodialysis, plasmapheresis, leukopheresis, extracorporeal membrane oxygenation (ECMO), heparin-induced extracorporeal membrane oxygenation LDL precipitation (HELP) and CPB. The factor B antibody, in accordance with the methods of the present invention, is believed to be useful in reducing or preventing the cognitive dysfunction that sometimes results from CPB procedures.

Published literature supports the use of simple models of extracorporeal circuits that mimic procedures such as CPB, dialysis, plasmapheresis in an effort to determine the efficacy of drugs to inhibit complications of such procedures. The model consists of tubing partially filled with blood and rotated at 37° C. to allow complement activation to occur at the blood polymer and the blood air interface, conditions of CPB. Blood circulation through artificial tubing surface of the clinical CPB or ECC circuit is a foreign surface that causes complement activation.

Ischemia Reperfusion Injury: This type of injury is most commonly found to be associated with alternative complement pathway activation. Ischemia reperfusion (I/R) injury occurs when a blood vessel is occluded naturally or by injury for an extended period of time, and the blood flow is restored after an extended period of ischemia. Blood re-perfusion in an ischemic tissue causes alternative pathway activation. This situation is found associated with aortic aneurysm repair, CPB, vascular re-anastomosis in connection with organ transplants (e.g., heart, lung, liver, kidney) and digit/extremity replantation, stroke, myocardial infarction and hemodynamic resuscitation following shock and/or surgical procedures. There are at least two major factors contributing to the ischemic insult: complement activation and cellular (neutrophil, monocyte, and platelet) activation.

Kidney I/R is an important cause of acute renal failure. The complement system appears to be essentially involved in renal I/R injury. In acute myocardial infarction (AMII), the plasma levels of Bb, and SC5b-9 increased only in patients with AMI.

Reperfusion of the ischemic myocardium during acute MI is required to prevent tissue damage, however, it is associated with downstream pathology that occurs after reperfusion. This so-called “reperfusion injury” is accompanied by a marked inflammatory reaction, which contributes to tissue injury. Oxygen free radicals and activation of neutrophils and monocytes in addition to activation of complement system represents the major contributors of the inflammatory reaction upon reperfusion. In fact, activation of the complement system may induce the activation of neutrophils and monocytes and generation of oxygen free radicals associated with reperfusion injury. As a result of complement activation, C3a, C5a and the membrane attack complex (MAC) are generated. C3a is known to activate monocytes, and C5a is known to activate neutrophils and both help attract neutrophils to the site of inflammation, leading to superoxide production. MAC is deposited over endothelial cells and smooth vessel cells, leading to tissue injury. An inhibitory drug that prevents C3a, C5a, and MAC production is expected to downregulate MI driven tissue injury.

Stroke: Stroke is a leading cause of morbidity and mortality in the United States. Recent animal studies have implicated the complement system in cerebral ischemia/reperfusion injury and suggest that complement inhibition may improve stroke outcomes.

Complement activation plays a role in contributing to inflammation within the injured brain. A role for complement activation in traumatic brain injury also was shown in C3 and C5 deficient mice. C3 and C5 defiency resulted in decreased neutrophil infiltration and reduced injury size after a traumatic brain injury. It has been shown that debris from injured neurons or myelin breakdown products trigger complement activation, including formation of C5b-9. Activated complement components may stimulate accumulation of inflammatory cells and formation of brain edema, as well as having membrane destructive effects by the end product, MAC, thereby being mediators in the development of secondary brain damage The complement pathways also play a role in the pathogenesis of acute brain injury which involves cerebral ischemia and trauma and chronic neurodegeneration such as Alzheimer's Disease. In animal model studies, complement C3 deficient mice demonstrated less brain edema and inflammatory response compared to C3 sufficient mice suggesting a role for complement in brain injury. One study evaluated the role of complement activation on cellular brain injury in patients undergoing coronary artery bypass grafting. The level of S100 beta, a marker of tissue injury, was found to be higher in patients with higher complement activation. These data suggested that activation of complement activation is related to neuronal injury.

Anaphylactic Shock: Anaphylactic shock, the most severe type of anaphylaxis, occurs when an allergic response triggers a quick release from mast cells of large quantities of immunological mediators (histamines, prostaglandins, leukotrienes) leading to systemic vasodilation (associated with a sudden drop in blood pressure) and edema of bronchial mucosa (resulting in bronchoconstriction and difficulty breathing). Reports have shown that anaphylaxis can be induced in isolated guinea pig hearts in the presence of complement. In these studies anaphylaxis was due to anaphylatoxin formation given that selected depletion of C3, which causes depletion of C3a and C5a formation, prevented anaphylaxis.

Atherosclerosis: Although no significant complement activation takes place in normal arteries, complement is extensively activated in atherosclerotic lesions especially in ruptured plaques. Components of the terminal complement pathway are frequently found in human atherosclerotic plaques. The extent of C5b-9 deposition was found to correlate with the severity of the lesion. Local complement activation may induce cell lysis and generate at least some of the cell debris found in the necrotic core of advanced lesions. Sublytic complement activation could be a significant factor contributing to monocyte infiltration into the arterial intima during atherogenesis. Persistent activation of complement may be detrimental because it may trigger and sustain inflammation. Complement inhibition by genetic C6 deficiency also has been shown to suppress the development of atherosclerosis without affecting serum cholesterol levels. One aspect of the invention is thus directed to the treatment or prevention of atherosclerosis by treating a subject suffering from or prone to atherosclerosis with a therapeutically effective amount of a factor B antibody in a pharmaceutical carrier.

Other Vascular Diseases and Conditions: There is evidence that complement activation contributes to the pathogenesis of many forms of vasculitis, including: Henoch-Schonlein purpura nephritis, systemic lupus erythematosus-associated vasculitis, vasculitis associated with rheumatoid arthritis (also called malignant rheumatoid arthritis), immune complex vasculitis, and Takayasu's disease. Henoch-Schonlein purpura nephritis is a form of systemic vasculitis of the small vessels with immune pathogenesis, in which activation of complement is recognized as an important mechanism. Complement activation also plays a role in dilated cardiomyopathy. Dilated cardiomyopathy is a syndrome characterized by cardiac enlargement and impaired systolic function of the heart. Recent data suggests that ongoing inflammation in the myocardium may contribute to the development of disease. C5b-9, the terminal membrane attack complex of complement, is known to significantly correlate with myocardial expression of TNF-alpha. In myocardial biopsies from 28 patients with dilated cardiomyopathy, myocardial accumulation of C5b-9 was demonstrated, suggesting that chronic immunoglobulin-mediated complement activation in the myocardium may contribute in part to the progression of dilated cardiomyopathy.

One aspect of this invention is directed to the treatment of a vascular condition, including cardiovascular conditions, cerebrovascular conditions, peripheral (e.g., musculoskeletal) vascular conditions, renovascular conditions, and mesenteric/enteric vascular conditions, by administering a composition comprising a therapeutically effective amount of a anti-factor B monoclonal antibodies in a pharmaceutical carrier. Conditions for which the invention is applicable include, without limitation: vasculitis, including Henoch-Schonlein purpura nephritis, systemic lupus erythematosus-associated vasculitis, vasculitis associated with rheumatoid arthritis (also called malignant rheumatoid arthritis), immune complex vasculitis, and Takayasu's disease; dilated cardiomyopathy; diabetic angiopathy; Kawasaki's disease (arteritis); and venous gas embolus (VGE). It is believed that the factor B antibody of the present invention may also be used in the inhibition of restenosis following stent placement, rotational atherectomy and/or percutaneous transluminal coronary angioplasty (PTCA), either alone or in combination with other restenosis inhibitory agents.

Following angioplasty, inflammatory mechanisms play a major role in the development of restenosis. The complement activation at the site is known to release C3a and C5a. The component, C5a, has strong chemotactic and proinflammatory effects. Enhanced complement activation prior to PTA, as measured by higher levels of C5a, was significantly associated with restenosis after SFA balloon angioplasty. Thus, inhibitors of complement activation may prove beneficial after angioplasty to down regulate inflammation.

Pulmonary Conditions: Complement plays a role in the pathogenesis of lung inflammatory disorders, such as acute respiratory distress syndrome (ARDS); transfusion-related acute lung injury (TRALI), ischemia/reperfusion acute lung injury, chronic obstructive pulmonary disease (COPD), asthma, Wegener's granulomatosis and antiglomerular basement membrane disease (Goodpasture's disease). All complement components can be produced locally in the lung by type II alveolar cells, alveolar macrophages and lung fibroblasts. Thus, the complement cascade can self perpetuate within lung and could lead to lung injury.

Patients with ARDS almost universally show evidence of extensive complement activation (increased plasma levels of complement components C3a and C5a), and the degree of complement activation has been correlated with the development and outcome of ARDS. In animal models, systemic activation of complement leads to acute lung injury with histopathology similar to that seen in human ARDS. In rat models, sCR1 has a protective effect in complement- and neutrophil-mediated lung injury.

Asthma is an inflammatory disease. The features of allergic asthma include airway hyperresponsiveness to a variety of specific and nonspecific stimuli, excessive airway mucus production, pulmonary eosinophilia, and elevated concentration of serum IgE. Although asthma appears to be multifactorial in origin, the fact that the complement system is highly activated in the human asthmatic lung is well documented. Many features of bronchial asthma, such as smooth muscle contraction, mucus secretion and recruitment of inflammatory cells, are consistent with the activation of complement because of elevated levels of C3a and C5a found associated with the disease. Production of C3a and C5a in asthmatic lung may be due to the activation of the alternative complement pathway on allergen surfaces or by the proteases released by inflammatory cells that could cleave the C3 and C5. The anaphylatoxins, C3a and C5a, are known to cause leukocyte activation, smooth muscle contraction and vascular permeability.

An aspect of the invention thus provides a method for treating pulmonary disorders, by administering a composition comprising a therapeutically effective amount of a factor Ba monoclonal antibody in a pharmaceutical carrier to a subject suffering from pulmonary disorders, including without limitation, acute respiratory distress syndrome, transfusion-related acute lung injury, ischemia/reperfusion acute lung injury, chronic obstructive pulmonary disease, asthma, Wegener's granulomatosis, antiglomerular basement membrane disease (Goodpasture's disease), meconium aspiration syndrome, bronchiolitis obliterans syndrome, idiopathic pulmonary fibrosis, acute lung injury secondary to burn, non-cardiogenic pulmonary edema, transfusion-related respiratory depression, and emphysema.

Transplantation: Complement activation significantly contributes to the inflammatory reaction following organ transplantation. In allotransplantation, the complement system may be activated by ischemia/reperfusion while in xenotransplantation the major activators for complement are pre-existing antibodies. Animal model studies have shown that the use of complement inhibitors may significantly prolong graft survival. Thus, there is an established role of the complement system in organ injury after organ transplantation. Alternative pathway mediated activation appears to mediate renal ischemia/reperfusion injury, and proximal tubular cells may be both the source and the site of attack of complement components in this setting. Locally produced complement in the kidney also plays a role in the development of both cellular and antibody-mediated immune responses against the graft.

Hyperacute graft rejection (HAR) triggered by the activation of the recipient's complement system represents the major obstacle to successful xenotransplantation. After the binding of preformed antibodies to vascular glycoproteins, complement-induced activation and injury of endothelial cells with subsequent thrombosis leads to rapid destruction of foreign tissues. Inhibition of complement activation is therefore considered as a prerequisite for xenograft (Xg) survival.

One aspect of the invention is thus directed to the prevention or treatment of inflammatory reaction resulting from tissue or solid organ transplantation by administering a composition comprising a therapeutically effective amount of an anti-factor Ba monoclonal antibody in a pharmaceutical carrier to the transplant recipient, including subjects that have received allotransplantation or xenotransplantation of whole organs (e.g., kidney, heart, liver, pancreas, lung, cornea, etc.) or grafts (e.g., valves, tendons, bone marrow, etc.).

Central and Peripheral Nervous System Disorders and Injuries: Activation of the complement system has been implicated in the pathogenesis of a variety of central nervous system (CNS) or peripheral nervous system (PNS) diseases or injuries, including but not limited to multiple sclerosis (MS), myasthenia gravis (MG), Huntington's disease (HD), amyotrophic lateral sclerosis (ALS), Guillain Barre syndrome, reperfusion following stroke, degenerative discs, cerebral trauma, traumatic neuronal injury, Parkinson's disease (PD) and Alzheimer's disease (AD). It has now been shown that C3a and C5a receptors are found on neurons and show widespread distribution in distinct portions of the sensory, motor and limbic brain systems

Multiple Sclerosis is characterized by a progressive loss of myelin ensheathing and insulating axons within the CNS. In both multiple sclerosis and EAE, complement activation is thought to play a pivotal role by recruiting inflammatory cells, increasing myelin phagocytosis by macrophages, and exerting direct cytotoxic effects through the deposition of the membrane attack complex on oligodendrocytes.

Activation of complement is critically involved in both Guillain-Barre syndrome (GBS) and multiple sclerosis (MS). Histological hallmarks of AD, are senile plaques and neurofibrillary tangles. The senile plaques stain strongly for components of the complement system. Evidence points to a local neuroinflammatory state that results in neuronal death and cognitive dysfunction. Chronic localized inflammation is an important element of AD pathogenesis that causes significant neurodegenerative damage. The amyloid beta peptide has been implicated as a primary activator of complement in AD. The peptide also localizes itself with drusen deposits categorizing them as sites of complement mediated inflammation. This suggests that amyloid deposition could be an important component of the local inflammatory events that contribute to atrophy of the retinal pigmented epithelium, drusen biogenesis, and the pathogenesis of age related macular degeneration. The inflammatory deposits also contain C3 and fragments suggesting activation of complement. The Alzheimer's A beta-peptide is deposited at sites of complement activation in pathologic deposits associated with aging and age-related macular degeneration. Drusen deposits are also hallmark of glomerular nephritis and age related macular degeneration. Elevated levels of C3, C5 and C5b-9 (MAC) have been found associated with the drunsen deposits.

In damaged regions in the brains of Parkinsons Disease (PD) patients, as in other CNS degenerative diseases, there is evidence of inflammation characterized by glial reaction (especially microglia), as well as increased expression of HLA-DR antigens, cytokines, and components of complement. These observations suggest that immune system mechanisms are involved in the pathogenesis of neuronal damage in PD. The cellular mechanisms of primary injury in PD have not been clarified, however, but it is likely that mitochondrial mutations, oxidative stress and apoptosis play a role. Furthermore, inflammation initiated by neuronal damage in the striatum and the substantial nigra in PD may aggravate the course of the disease. These observations suggest that treatment with complement inhibitory drugs may act to slow progression of PD.

Significantly higher C3b concentrations in patients with active dermatomyositis, Guillain-Barré syndrome and myasthenia gravis, compared to inclusion body myositis and controls is suggestive of complement activation. The in-vitro C3 uptake assay supports the role of C3b neoantigen and membranolytic attack complex deposition in the target tissues and may be a useful tool to monitor disease activity in patients with complement-mediated neurological disorders.

One aspect of the invention is thus directed to the treatment of peripheral nervous system (PNS) and/or central nervous system (CNS) disorders or injuries by treating a subject suffering from such a disorder or injury with a composition comprising a therapeutically effective amount of an anti-factor Ba monoclonal antibody inhibitory agent in a pharmaceutical carrier. CNS and PNS disorders and injuries that may be treated in accordance with the present invention are believed to include but are not limited to multiple sclerosis (MS), myasthenia gravis (MG), Huntington's disease (HD), amyotrophic lateral sclerosis (ALS), Guillain Barre syndrome, reperfusion following stroke, degenerative discs, cerebral trauma, Parkinson's disease (PD), Alzheimer's disease (AD), Miller-Fisher syndrome, cerebral trauma and/or hemorrhage, demyelination and, possibly, meningitis.

Blood Disorders: Complement activation has been shown in numerous studies to have a major role in the pathogenesis of sepsis. Sepsis is usually defined as the systemic host response to an infection. However, on many occasions, no clinical evidence for infection (e.g., positive bacterial blood cultures) is found in patients with septic symptoms. This discrepancy was first taken into account at a Consensus Conference in 1992 when the term “systemic inflammatory response syndrome” (SIRS) was established, and for which no definable presence of bacterial infection was required. Many studies have shown the importance of complement activation in mediating inflammation and contributing to the features of shock, particularly septic and hemorrhagic shock. Both Gram-negative and Gram-positive organisms commonly precipitate septic shock. The major components of the Gram-positive cell wall are peptidoglycan and lipoteichoic acid, and both components are potent activators of the alternative complement pathway. The complement system was initially implicated in the pathogenesis of sepsis when it was noted by researchers that the anaphylatoxins C3a and C5a mediate a variety of inflammatory reactions that might also occur during sepsis. These anaphylatoxins evoke vasodilation and an increase in microvascular permeability, events that play a central role in septic shock. In addition, the anaphylatoxins induce bronchospasm, histamine release from mast cells, and aggregation of platelets. Moreover, they exert numerous effects on granulocytes, such as chemotaxis, aggregation, adhesion, release of lysosomal enzymes, generation of toxic super oxide anions and formation of leukotrienes. These biologic effects are thought to play a role in development of complications of sepsis such as shock or acute respiratory distress syndrome (ARDS).

An aspect of the invention thus provides a method for treating sepsis or a condition resulting from sepsis, by administering a composition comprising a therapeutically effective amount of a anti-factor Ba antibody in a pharmaceutical carrier to a subject suffering from sepsis or a condition resulting from sepsis including without limitation severe sepsis, septic shock, acute respiratory distress syndrome resulting from sepsis, and systemic inflammatory response syndrome. Related methods are provided for the treatment of other blood disorders, including hemorrhagic shock, hemolytic anemia, autoimmune thrombotic thrombocytopenic purpura (TTP), hemolytic uremic syndrome (HUS) or other marrow/blood destructive conditions, by administering a composition comprising a therapeutically effective amount of an anti-factor B monoclonal antibody inhibitory agent in a pharmaceutical carrier to a subject suffering from such a condition.

Inflammatory and Non-Inflammatory Arthritides and Other Musculoskeletal Diseases: Complement activation has been implicated in the pathogenesis of a wide variety of chronic diseases; including rheumatoid arthritis (RA), juvenile rheumatoid arthritis, osteoarthritis, systemic lupus erythematosis (SLE), Behcet's syndrome and Sjogren's syndrome. There are several publications documenting that complement activation products (C3a, C5a, and C5b-9) are elevated in the plasma of RA patients. Complement activation products have also been found within inflamed rheumatic joints. Positive correlations have been established between the degree of complement activation and the severity of RA. In both adult and juvenile rheumatoid arthritis, elevated serum and synovial fluid levels of Bb compared to C4d (a marker for classical pathway activation), clearly suggest that complement activation in RA is mediated predominantly via the alternative pathway. There is compelling evidence that immune-complex-triggered complement activation is a major pathological mechanism that contributes to tissue damage in rheumatoid arthritis (RA). Immune complex-mediated activation of complement through the classic pathway is believed to be one mechanism by which tissue injury occurs in RA patients. However, the amplification loop of the alternative pathway is required for classical pathway activation and propagation.

Animal models of experimental arthritis have been widely used to investigate the role of complement in the pathogenesis of RA. Systemic lupus erythematosus (SLE) is an autoimmune disease of undefined etiology that results in production of autoantibodies, generation of circulating immune complexes, and episodic, uncontrolled activation of the complement system. Although the origins of autoimmunity in SLE remain elusive, considerable information is now available implicating complement activation as an important mechanism contributing to vascular injury in this disease. Activation of both the classical and alternative pathways of complement is involved in the disease. Both C4d and Bb are sensitive markers of moderate-to-severe lupus disease activity. Activation of the alternative complement pathway accompanies disease flares in SLE during pregnancy. In addition, the lectin pathway may contribute to disease development since autoantibodies against MBL have recently been identified in sera from SLE patients. Immune complex-mediated activation of complement through the classic pathway is believed to be one mechanism by which tissue injury occurs in SLE patients. Complement activation may be an important mechanism contributing to SLE pathogenesis. The alternative pathway also has an important role in the autoimmune disease manifestations of SLE since backcrossing of factor B-deficient mice onto the MRL/1 pr model of SLE revealed that the lack of factor B lessened the vasculitis and glomerular disease. SLE is an example of systemic autoimmune diseases that affects multiple organs, including skin, kidneys, joints, serosal surfaces, and the CNS. SLE is frequently associated with severe vasculitis. Activated complement components appear in the circulation of patients with SLE, Elevated levels of C3a and C5a were identified in SLE patients. That these proinflammatory molecules are elevated during exacerbation of the disease suggest they may contribute to the vascular injury in SLE patients. Horiquome et al showed that alternative complement pathway is activated in membranoproliferative glomerulonephritis (MPGN) as suggested by the measurement of alternative pathway depenedent hemolysis of the patient plasma. In a study using the cobra venom factor to deplete complement components, benefits in the disease were noticed in that glomerular deposits of complement C3b (C3c) deposits cleared within 24 hours of cessation of complement activation.

Results from both human and animal studies support the possibility that the complement system contributes directly to the pathogenesis of muscular dystrophy. Studies of human dystrophic biopsies have shown that C3 and C9 are deposited on both necrotic and non-necrotic fibers in dystrophic muscle. Using DNA microarray methods, Porter and colleagues found markedly enhanced gene expression of numerous complement-related mRNAs in dystrophin-deficient (mdx) mice coincident with development of the dystrophic disease.

One aspect of the invention is for the prevention and/or treatment of inflammatory and non-inflammatory arthritides and other musculoskeletal disorders, including but not limited to osteoarthritis, rheumatoid arthritis juvenile rheumatoid arthritis, gout, neuropathic arthropathy, psoriatic arthritis, ankylosing spondylitis or other spondyloarthropathies and crystalline arthropathies, muscular dystrophy or SLE, by administering a composition comprising a therapeutically effective amount of a anti-factor B monoclonal antibody inhibitory agent in a pharmaceutical carrier to a subject suffering from such a disorder.

Renal Conditions: Activation of the complement system has been implicated in the pathogenesis of a wide variety of renal diseases; including, mesangioproliferative glomerulonephritis (IgA-nephropathy, Berger's disease), membranous glomerulonephritis, membranoproliferative glomerulonephritis (mesangiocapillary glomerulonephritis), acute postinfectious glomerulonephritis (poststreptococcal glomerulonephritis), cryoglobulinemic glomerulonephritis, lupus nephritis, and Henoch-Schonlein purpura nephritis. Kahn and Sinniah demonstrated increased deposition of C5b-9 in tubular basement membranes in biopsies taken from patients with various forms of glomerulonephritis. Another study of membranous nephropathy demonstrated a positive correlation between increased sC5b-9 levels and poor prognosis. These various studies suggest that ongoing complement-mediated glomerulonephritis results in urinary excretion of complement proteins that correlate with the degree of tissue damage and disease prognosis. One aspect of the invention is thus directed to the treatment of renal conditions including but not limited to mesangioproliferative glomerulonephritis, membranous glomerulonephritis, membranoproliferative glomerulonephritis (mesangiocapillary glomerulonephritis), acute postinfectious glomerulonephritis (poststreptococcal glomerulonephritis), cryoglobulinemic glomerulonephritis, lupus nephritis, Henoch-Schonlein purpura nephritis or IgA nephropathy, by administering a composition comprising a therapeutically effective amount of a anti-factor B monoclonal antibody inhibitory agent in a pharmaceutical carrier to a subject suffering from such a disorder.

Ulcerative colitis and Crohn's disease are chronic inflammatory disorders of the bowel that fall under the banner of inflammatory bowel disease (IBD). IBD is characterized by spontaneously occurring, chronic, relapsing inflammation of unknown origin. Despite extensive research into the disease in both humans and experimental animals, the precise mechanisms of pathology remain to be elucidated. However, the complement system is believed to be activated in patients with IBD and is thought to play a role in disease pathogenesis. It has been shown that C3b and other activated complement products are found at the luminal face of surface epithelial cells, as well as in the muscularis mucosa and submucosal blood vessels in IBD patients. Furthermore, polymorphonuclear cell infiltration, usually a result of C5a generation, characteristically is seen in the inflammatory bowel. A novel human C5a receptor antagonist has been shown to protect against disease pathology in a rat model of IBD.

Complement activation plays a critical role in acute pancreatitis. In pancreatitis patients, significantly elevated levels of C3a, C5a and MAC were found compared to controls. As a result, down stream events such as increased vascular permeability, anemia and impaired respiration in these patients may be influenced by complement activation. Anaphylatoxins and terminal complement complexes in pancreatitis. Evidence of complement activation in plasma and ascites fluid of patients with acute pancreatitis. In additional studies, levels of C3a and C5a correlated to the severity of the disease.

Studies by Gloor et al has tested the presence of complement activation byproducts C3a and sC5b-9 and demonstrated that these were significantly elevated during the first 7 days in plasma of patients with severe acute pancreatitis, as compared to control patients. Pancreatic enzymes release proteases that cause activation of complement resulting in elevated levels of C3a and C5a. In the setting of animal model studies on experimental acute pancreatitis, the sera demonstrated excessive complement activation and neutrophil lung sequestration, an early event in acute pancreatitis. In sCR1 treated animals, the total complement activation was down regulated including neutrophil activation as indicated by the downregulation of CD11b expression.

The present invention thus provides methods for inhibiting factor Ba dependent complement activation in subjects suffering from inflammatory gastrointestinal disorders, including but not limited to pancreatitis, diverticulitis and bowel disorders including Crohn's disease, ulcerative colitis, and irritable bowel syndrome, by administering a composition comprising a therapeutically effect amount of a anti-factor Ba monoclonal antibody inhibitory agent in a pharmaceutical carrier to a patient suffering from such a disorder.

Skin Disorders: The underlying etiology of various skins diseases such as psoriasis support a role for immune and proinflammatory processes including the involvement of the complement system. Its activation leads to the generation of products that not only help to maintain normal host defenses, but also mediate inflammation and tissue injury. Proinflammatory products of complement include C3a, C4a, and C5a, and membrane attack complexes. Among them, C5a or its degradation product C5a des Arg, seems to be the most important mediator because it exerts a potent chemotactic effect on inflammatory cells. Intradermal administration of C5a anaphylatoxin induces skin changes quite similar to those observed in cutaneous hypersensitivity vasculitis that occurs through immune complex-mediated complement activation.

While the classical pathway of the complement system has been shown to be activated in psoriasis, there are fewer reports regarding involvement of the alternative pathway in the inflammatory reactions in psoriasis. Complement activation in psoriatic lesional skin also results in the deposition of terminal complement complexes within the epidermis as defined by measuring levels of SC5b-9 in the plasma and horny tissues of psoriatic patients. The levels of sC5b-9 in psoriatic plasma have been found to be significantly higher than those of controls. There is a significant activation of the alternative pathway following thermal injury. In thermal injury, chronic inflammation sets in if the injury remains untreated. The evidence for alternative pathway activation comes from the studies that determined the presence of elevated levels of complement activation byproducts in the plasma of burn patients.

One aspect of the invention is thus directed to the treatment of psoriasis, autoimmune bullous dermatoses, eosinophilic spongiosis, bullous pemphigoid, epidermolysis bullosa acquisita, atopic dermatitis, herpes gestationis and other skin disorders, and for the treatment of thermal and chemical burns including capillary leakage caused thereby, by administering a composition comprising a therapeutically effective amount of a anti-factor B antibody inhibitory agent in a pharmaceutical carrier to a subject suffering from such a skin disorder.

Ophthalmologic Conditions: Gerl et al. determined the presence of activated complement components in eyes affected by diabetic retinopathy. Immunohistochemical studies found extensive deposits of complement C5b-9 complexes that were detected in the choriocapillaris immediately underlying the Bruch membrane and densely surrounding the capillaries in all 50 diabetic retinopathy specimens examined. Staining for C3d positively correlated with C5b-9 staining, indicative of the fact that complement activation had occurred in situ. Complement activation may be a causative factor in the pathologic sequelae that can contribute to ocular tissue disease and visual impairment. Therefore, the use of a complement inhibitor may be an effective therapy to reduce or block damage to microvessels that occurs in diabetes.

Age-related macular degeneration (AMD) results in the progressive destruction of the macula. Destruction of the macula in AMD has been correlated with the formation of extracellular deposits called drusen located in and around the macula, behind the retina and between the retina pigment epithelium (RPE) and the choroids. Identification and localization of complement activation products (C3a, C5a, C3b, C5b-9) have led investigators to conclude that chronic complement activation plays an important role in the process of drusen biogenesis and the etiology of AMD. Immunostaining studies suggest that complement activation in drusen does not occur via the classical pathway. Using immunohistological methods, Bora and colleagues (2005) found significant deposition of the complement activation products C3b and C5b-9 (MAC) in the neovascular complex (CNV) following laser treatment. Previous studies have established that recruitment of leukocytes and macrophages in particular, plays a pivotal role in laser-induced CNV. In their 2006 paper, Nozaki and colleagues report that leukocyte recruitment is markedly reduced in C3aR(−/−) and C5aR(−/−) mice after laser injury.

An aspect of the invention thus provides a method for inhibiting factor Ba-dependent complement activation to treat age-related macular degeneration or other complement mediated ophthalmologic conditions by administering a composition comprising a therapeutically effective amount of a anti-factor Ba monoclonal antibody inhibitory agent in a pharmaceutical carrier to a subject suffering from such a condition or other complement-mediated ophthalmologic condition.

Radiographic Contrast Media: The direct effect of four different radiographic contrast media (RCM) on the release of C3a and C5a and the production of IL-1 alpha and TNF-alpha from vascular endothelial cells was examined in vitro. An aspect of the invention thus provides a method for inhibiting factor Ba-dependent complement activation to treat inflammation as a result of RCM or other complement mediated inflammatory condition by administering a composition comprising a therapeutically effective amount of a anti-factor B monoclonal antibody inhibitory agent in a pharmaceutical carrier to a subject suffering from such a condition or other complement-mediated RCM condition.

### Treatment Methods

The methods generally involve administering to a mammalian subject in need thereof an effective amount of a subject antibody for including methods of reducing the level of a polypeptide generated following activation of the alternative complement pathway; methods of reducing the level of membrane attack complex (MAC); methods of reducing the level of an anaphylatoxin; methods of reducing the level of C3c; and methods of treating a disease or disorder mediated by the alternative complement pathway.

An “effective amount” of a subject antibody is an amount that is effective to reduce the production and/or level of a polypeptide generated following activation of the alternative complement pathway by at least about 20%, at least about 30%, at least about 40%, at least about 50%, at least about 60%, at least about 70%, at least about 80%, at least about 90%, or more.

A subject antibody is administered to an individual in a formulation with a pharmaceutically acceptable excipient(s). A wide variety of pharmaceutically acceptable excipients are known in the art and need not be discussed in detail herein.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In the subject methods, a subject antibody may be administered to the host using any convenient means capable of resulting in the desired therapeutic effect. Thus, the antibody can be incorporated into a variety of formulations for therapeutic administration. More particularly, a subject antibody can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of a subject antibody can be achieved in various ways, including oral, buccal, rectal, parenteral, intraperitoneal, intradermal, subcutaneous, intramuscular, transdermal, intranasal, pulmonary, intratracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

A subject antibody can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of a subject antibody calculated in an amount sufficient to produce the desired effect in association with a pharmaceutically acceptable diluent, carrier or vehicle.

A subject antibody is administered to an individual at a frequency and for a period of time, so as to achieve the desired therapeutic effect. For example, a subject antibody is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), or substantially continuously, or continuously, over a period of time ranging from about one day to about one week, from about two weeks to about four weeks, from about one month to about two months, from about two months to about four months, from about four months to about six months, or longer.

### Combination Therapy

The anti-factor Ba antibody will in some embodiments be administered in an effective amount in combination therapy with a second therapeutic agent. Suitable second therapeutic agents include, but are not limited to, anti-inflammatory agents; agents used for the treatment of cardiovascular disorders; steroidal anti-inflammatory agents; and the like.

Suitable anti-inflammatory agents include, but are not limited to, non-steroidal anti-inflammatory drugs (NSAIDs) acetaminophen, salicylate, acetyl-salicylic acid (aspirin, diflunisal), ibuprofen, Motrin, Naprosyn, Nalfon, and Trilisate, indomethacin, glucametacine, acemetacin, sulindac, naproxen, piroxicam, diclofenac, benoxaprofen, ketoprofen, oxaprozin, etodolac, ketorolac tromethamine, ketorolac, nabumetone, and the like, and mixtures of two or more of the foregoing. Other suitable anti-inflammatory agents include methotrexate.

Suitable steroidal anti-inflammatory agents include, but are not limited to, hydrocortisone, prednisone, prednisolone, methylprednisolone, dexamethasone, betamethasone, and triamcinolone.

Examples agents for cardiovascular indications include GP IIb-IIIa inhibitors such as INTEGRILIN (eptifibatide); aprotinin; REOPRO (abciximab); and the like.

Suitable second therapeutic agents include beta adrenergics which include bronchodilators including albuterol, isoproterenol sulfate, metaproterenol sulfate, terbutaline sulfate, pirbuterol acetate and salmeterol formotorol; steroids including beclomethasone dipropionate, flunisolide, fluticasone, budesonide and triamcinolone acetonide.

Anti-inflammatory drugs used in connection with the treatment of respiratory diseases include steroids such as beclomethasone dipropionate, triamcinolone acetonide, flunisolide and fluticasone. Other examples of anti-inflammatory drugs include cromoglycates such as cromolyn sodium. Other respiratory drugs, which would qualify as bronchodilators, include anticholenergics including ipratropium bromide. Antihistamines include, but are not limited to, diphenhydramine, carbinoxamine, clemastine, dimenhydrinate, pryilamine, tripelennamine, chlorpheniramine, brompheniramine, hydroxyzine, cyclizine, meclizine, chlorcyclizine, promethazine, doxylamine, loratadine, and terfenadine. Particular

anti-histamines include rhinolast (Astelin), claratyne (Claritin), claratyne D (Claritin D), telfast (Allegra), zyrtec, and beconase.

In some embodiments, the anti-factor Ba antibody is administered concurrently with a second therapeutic agent. As used herein, the term “concurrently” indicates that the subject antibody and the second therapeutic agent are administered separately and are administered within about 5 seconds to about 15 seconds, within about 15 seconds to about 30 seconds, within about 30 seconds to about 60 seconds, within about 1 minute to about 5 minutes, within about 5 minutes to about 15 minutes, within about 15 minutes to about 30 minutes, within about 30 minutes to about 60 minutes, within about 1 hour to about 2 hours, within about 2 hours to about 6 hours, within about 6 hours to about 12 hours, within about 12 hours to about 24 hours, or within about 24 hours to about 48 hours of one another.

In some embodiments, the anti-factor Ba antibody is administered during the entire course of treatment with the second therapeutic agent. In other embodiments, a subject antibody is administered for a period of time that is overlapping with that of the treatment with the second therapeutic agent, e.g., the antibody treatment can begin before the treatment with the second therapeutic agent begins and end before the treatment with the second therapeutic agent ends; the antibody treatment can begin after the treatment with the second therapeutic agent begins and end after the antibody treatment ends; the antibody treatment can begin after the treatment with the second therapeutic agent begins and end before the treatment with the second therapeutic agent ends; or antibody treatment can begin before the treatment with the second therapeutic agent begins and end after the treatment with the second therapeutic agent ends.

### Subjects for Treatment

Subjects that can be treated with the anti-factor Ba antibody and combination therapies of the present invention include individuals suffering from one or more of the following disorders: atherosclerosis, ischemia-reperfusion following acute myocardial infarction, Henoch-Schonlein purpura nephritis, immune complex vasculitis, rheumatoid arthritis, arteritis, aneurysm, stroke, cardiomyopathy, hemorrhagic shock, crush injury, multiple organ failure, hypovolemic shock and intestinal ischemia, transplant rejection, cardiac Surgery, PTCA, spontaneous abortion, neuronal injury, spinal cord injury, myasthenia gravis, Huntington‘s disease, amyotrophic lateral sclerosis, multiple sclerosis, Guillain Barre syndrome, Parkinson's disease, Alzheimer's disease, acute respiratory distress syndrome, asthma, chronic obstructive pulmonary disease, transfusion-related acute lung injury, acute lung injury, Goodpasture’ s disease, myocardial infarction, post-cardiopulmonary bypass inflammation, cardiopulmonary bypass, septic shock, transplant rejection, xeno transplantation, burn injury, systemic lupus erythematosus, membranous nephritis, Berger's disease, psoriasis, pemphigoid, dermatomyositis, anti-phospholipid syndrome, inflammatory bowel disease, hemodialysis, leukopheresis, plasmapheresis, heparin-induced extracorporeal membrane oxygenation LDL precipitation, extracorporeal membrane oxygenation, and macular degeneration.

In an a particular aspect of the invention, subjects that can be treated with a subject method include individuals suffering from one or more of the following disorders:

post-cardiopulmonary bypass inflammation, myocardial infarction, stroke, acute respiratory distress syndrome (ARDS), septic shock, transplant rejection, bum injury, multiple sclerosis, myasthenia gravis, cardiovascular disorders, and rheumatoid arthritis. Subjects suitable for treatment with a subject method also include individuals suffering from any inflammatory disorder, including, but not limited to, systemic lupus erythematosus, membranous nephritis, pemphigoid, dermatomyositis, and anti-phospholipid syndrome. Subjects suitable for treatment also include subjects undergoing renal dialysis.

### EXAMPLES

The “Examples” which follow are presented to describe preferred embodiments and utilities of the invention and are not meant to limit the invention unless otherwise stated in the claims appended hereto. The description is intended as a non-limiting illustration, since many variations will become apparent to those skilled in the art in view thereof. It is intended that all such variation within the scope and spirit of the appended claims be embraced thereby. Changes can be made in the composition, operation, and arrangement of the method of the present invention described herein without departing from the concept and scope of the invention as defined in the claims.

### Example 1

### Schematics Showing the Classical, the Lectin and the Alternative Complement Pathway

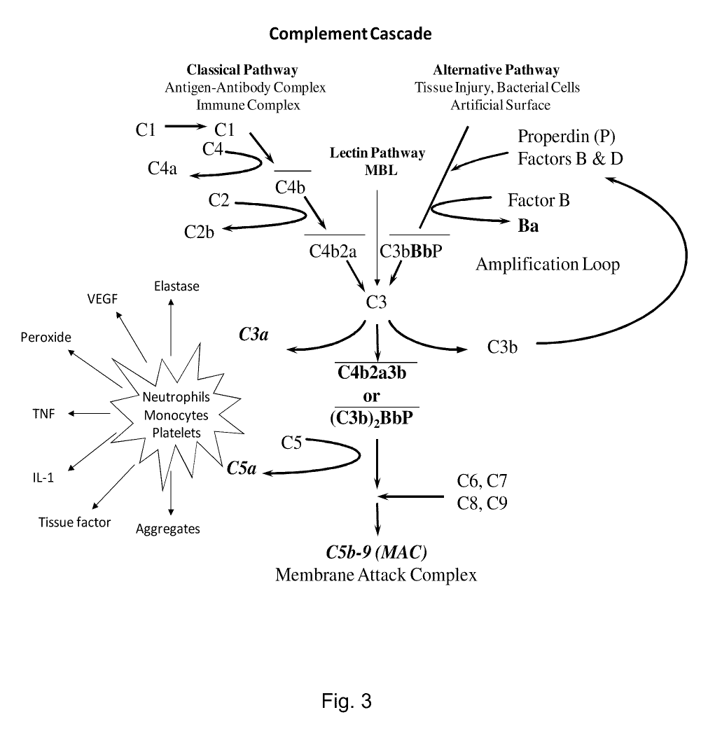

As shown, FIG. 3 both the classical pathway and the alternative pathway converge at C3. The C3 molecule is split into C3a and C3b by the action of C3 convertases. As the activation proceeds, C5a and C5b-9 are formed. Both C3a and C5a activate neutrophils, monocytes and platelets. Activated cells release inflammatory molecules.

### Example 2

### Binding of Factor B to C3b and Properdin-Bound C3b

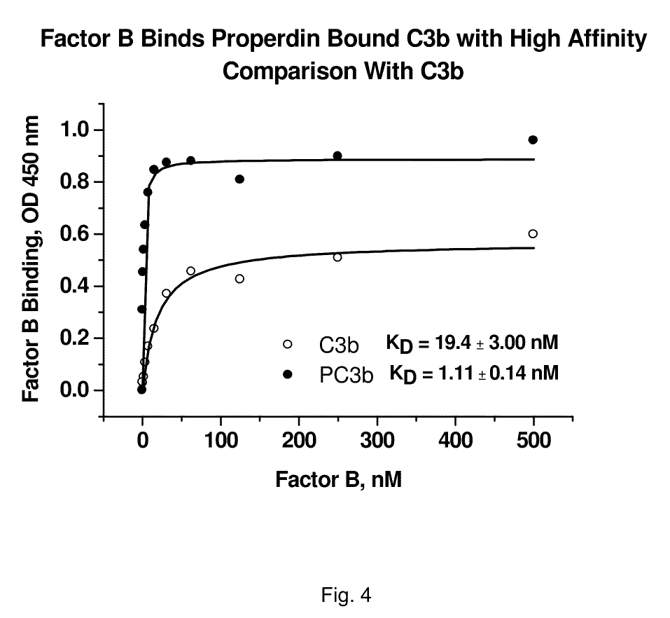

Polystyrene microtiter plates were coated with human C3b in phosphate buffered saline (PBS) overnight at 4 degree. After aspirating the C3b solution, wells were blocked with PBS containing 1% bovine serum albumin (BSA) for 2 hours at room temperature. Wells without C3b coating served as background controls. Aliquots of human factor B with and without properdin were added and plates were allowed to sit for 2 hours to the C3b coated wells to allow properdin and factor B binding. Following 2-hour incubation at room temperature, the wells were extensively rinsed with PBS and Factor B bound to properdin-bound C3b and C3b was detected by the addition of mouse monoclonal anti-human factor B antibody (detection antibody) at 1:1000 dilution in blocking solution, which was allowed to incubate for 1 hour at room temperature. After washing the plates with PBS, a peroxidase-conjugated goat anti-mouse antibody was added and allowed to incubate for 1 hour. The plate was again rinsed thoroughly with PBS, and 100 μl of 3,3′,5,5′-tetramethyl benzidine (TMB) substrate was added. After incubation for 10 minutes at 25° C., the reaction of TMB was quenched by the addition of 100 μl of phosphoric acid, and the plate was read at 450 nm in a microplate reader The estimated Kd of factor B binding to C3b or P-C3b were determined. As shown in FIG. 4, human factor B binds to both Properdin-bound C3b and C3b, which has been immobilized onto microtiter plate wells. The affinity of factor B binding to properdin bound C3b was higher than C3b. These data suggest that factor B preferentially binds properdin bound C3b.

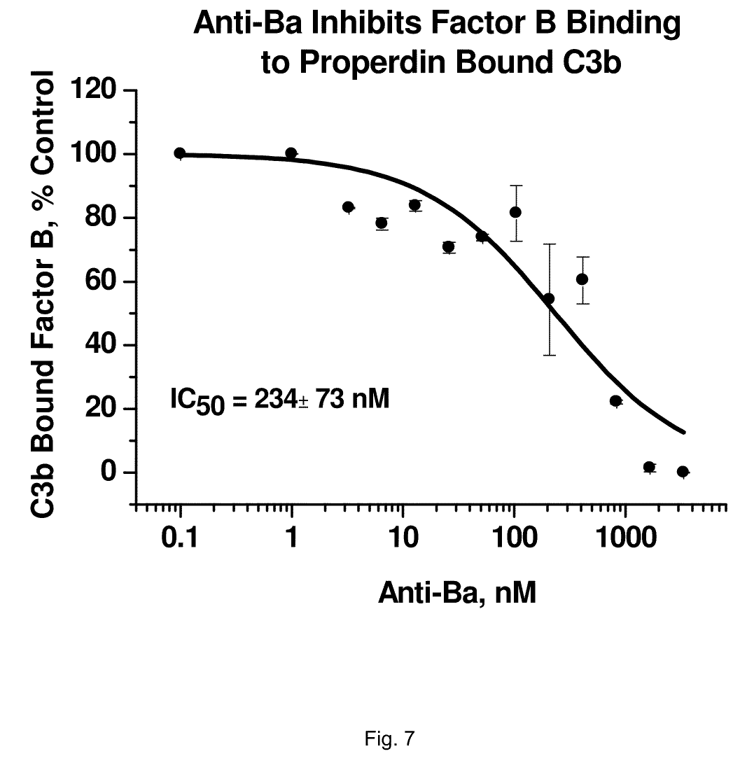

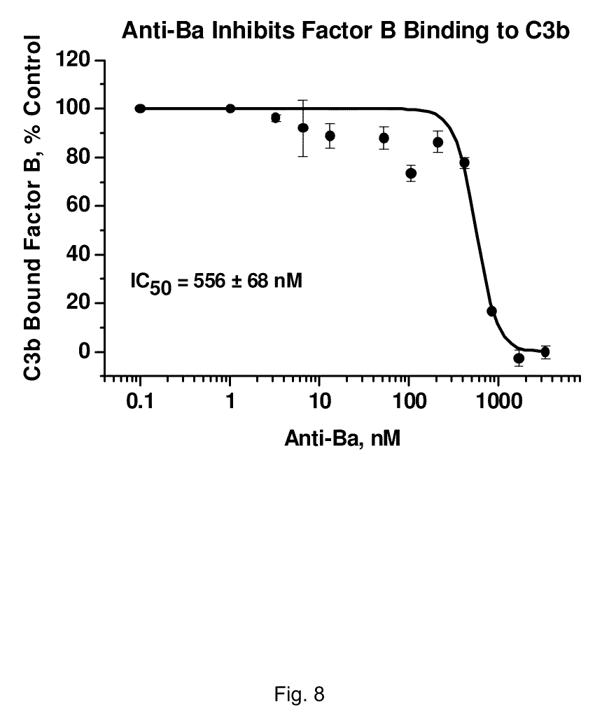

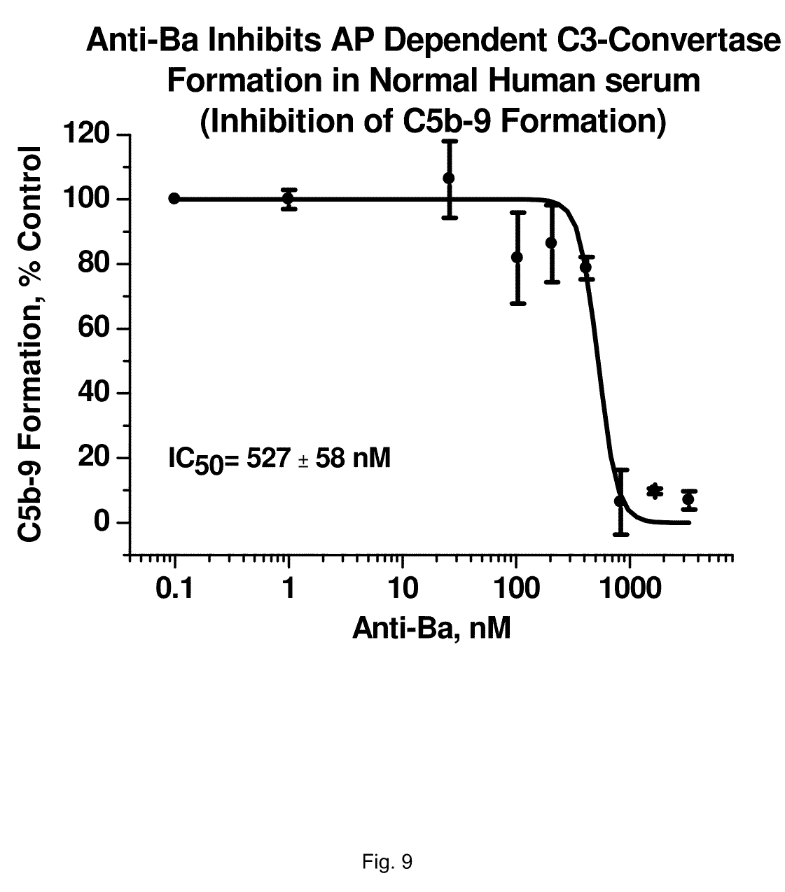

In a separate experiment, to demonstrate the effect of Anti-Ba on inhibition of Factor B binding to substrate-bound C3b or P-C3b complex, Anti-Ba was added to the factor B solution with and without properdin. The assay was repeated as above. FIG. 7 and 8 show that Anti-Ba inhibits factor B binding to C3b or properdin bound C3b with an IC50 of 200-500 nM.

### Example 3

### Factor B Does Not Bind iC3b, Cc, and C3dg With and Without Properdin

Polystyrene microtiter plates were coated with human iC3b, C3c, and C3dg in PBS overnight. After aspirating the respective solutions, wells were blocked with PBS containing 1% BSA for 2 hours at room temperature. Aliquots of human factor B at varying concentrations in blocking solution were added to the wells. Following 2-hour incubation at room temperature, the wells were extensively rinsed with PBS.

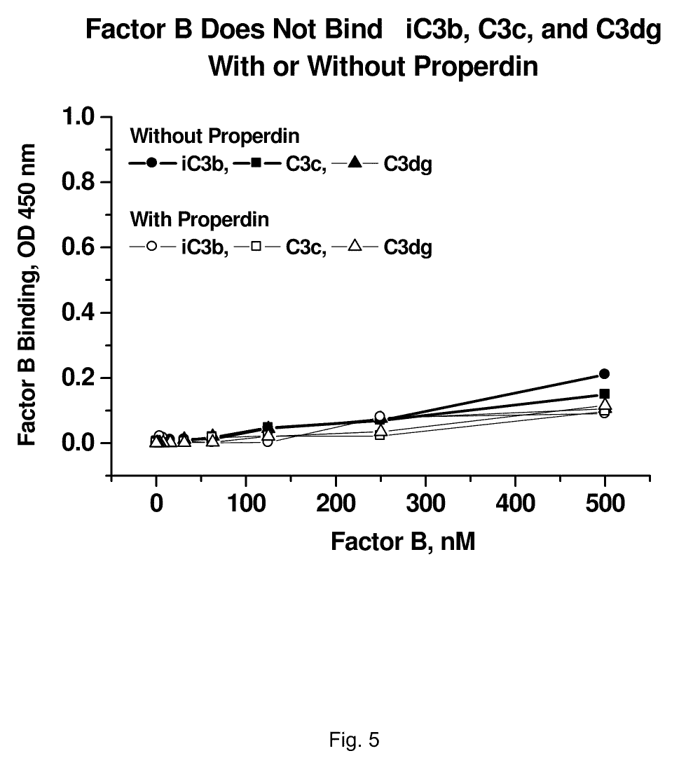

Protein-bound B was detected by the addition of mouse monoclonal anti-human factor B antibody (detection antibody) at 1:1000 dilution in blocking solution, which was allowed to incubate for 1 hour at room temperature. After washing the plates with PBS, a peroxidase-conjugated goat anti-mouse antibody was added and allowed to incubate for 1 hour. The plate was again rinsed thoroughly with PBS, and 3,3′,5,5′-tetramethyl benzidine (TMB) substrate was added. After incubation for 10 minutes at 25° C., the reaction of TMB was quenched by the addition of phosphoric acid, and the plate was read at 450 nm in a microplate reader as shown in FIG. 5, human factor Ba does not bind iC3b, C3c and C3dg.

### Example 4

### Anti-Ba Selectively Binds Ba Protein Not Bb Protein

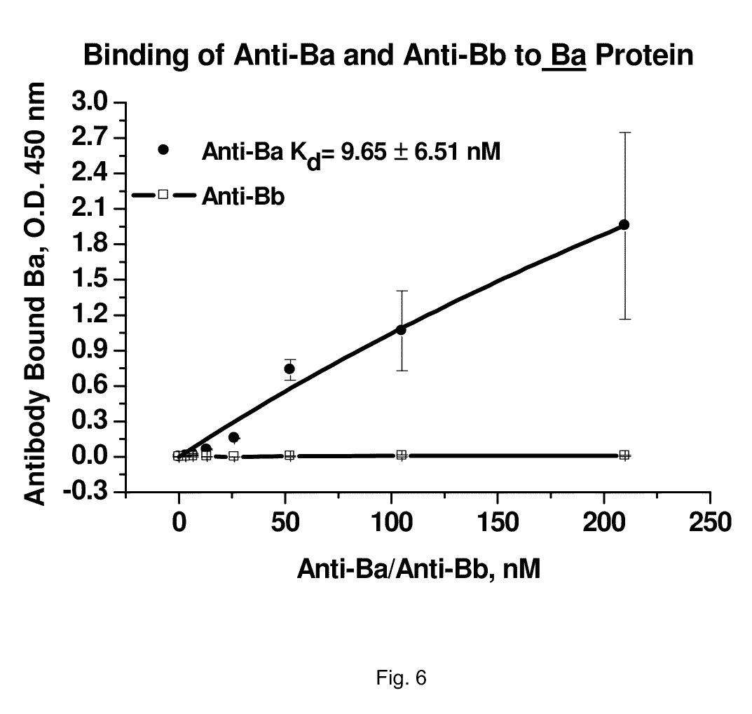

Anti-Ba binds the Ba protein without binding the Bb protein and therefore is a selective monoclonal for Ba. To determine the binding affinity, polystyrene microtiter wells were coated with Ba protein overnight at 4 degree. Wells without Ba coating served as background controls. After removal of the Ba solution, the wells were blocked with 1% BSA-PBS solution for 1-hour. Following blocking, the wells were incubated with 100 μl of increasing concentrations of Anti-Ba antibodies in their respective wells. The antibodies were incubated at room temperature for 1-hour and then aspirated and washed with PBS. The bound antibodies were detected with 100 μl of a peroxidase conjugated goat anti-mouse antibody at 1:2000 dilution for 1-hour at room temperature. Following incubation, the wells were aspirated and washed with PBS. The reaction was developed by the addition of 100 μl of TMB substrate to each well. The reaction of TMB was quenched by the addition of 100 μl of 1 M phosphoric acid and the plate was read at 450 nm with a microplate reader using SoftMax Pro 3.1.2 software. The data from three independent experiments were averaged to determine the error bars. As shown in FIG. 6, Anti-Ba only binds the Ba protein.

### Example 5

### Anti-Ba Inhibits C5b-9 Formation in Human Serum

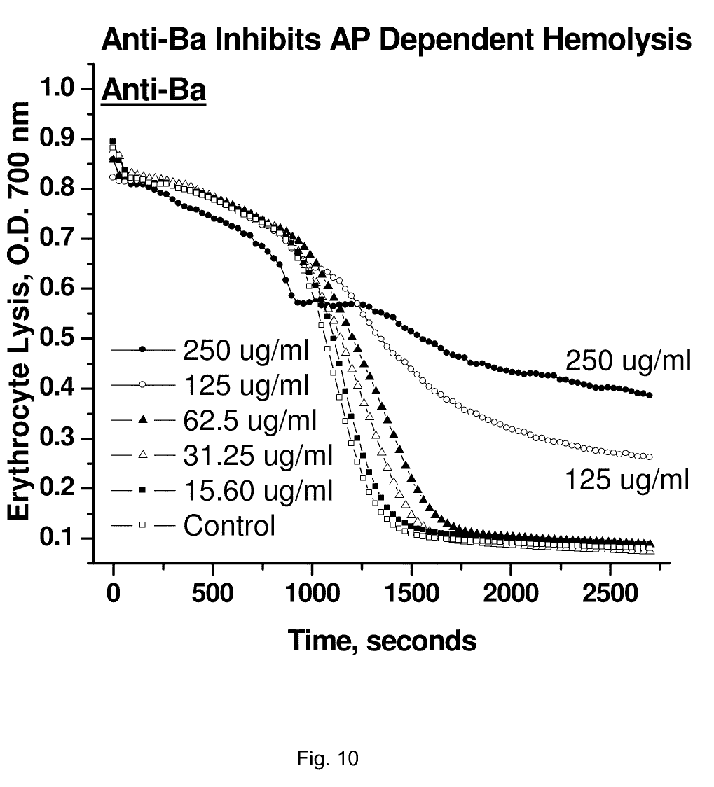

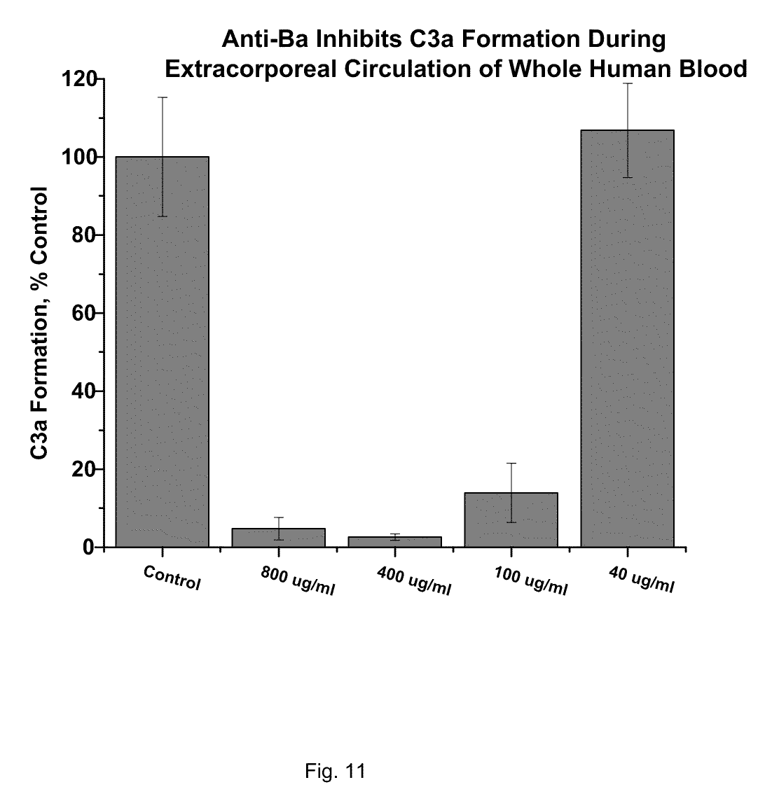

This experiment shows that the generation of C5b-9 in human serum is inhibited by the addition of an anti-factor Ba monoclonal antibody. Wells of microtiter plates were coated with LPS 2 μg/50 μl in PBS. The wells were incubated with 1% BSA in PBS to block the unoccupied sites on the plate. Normal human serum (10%) with and without anti-factor Ba monoclonal antibody was incubated in the wells. The plate was warmed to 37° C. for 2 hours to allow complement activation to occur. Following the incubation, the plate was washed and the deposited C5b-9 was detected with appropriate antibodies. As shown in the FIG. 10, Anti-Ba inhibits C5b-9 formation in a dose dependent manner with an IC₅₀ of 527 nM.

### Example 6

### Anti-Ba Inhibits Alternative Pathway-Dependent Hemolysis of Erythrocytes

The effect of anti-factor Ba antibody was examined in another assay of the alternative pathway. Rabbit erythrocytes initiate the alternative complement cascade, and the resulting formation of C5b-9 causes lysis of these cells. In this assay, normal human serum, at various concentrations in Gelatin Veronal Buffer (GVB) with 5 mM magnesium chloride and 10 mM EGTA, was incubated at 37° C. with a fixed number of rabbit erythrocytes (Advanced Research Technology). A progressive decrease in light scatter (due to lysis of intact cells) was measured at 700 nm as a function of time in a temperature-controlled ELISA plate reader. To determine the ability of blocking antibody to inhibit hemolysis of rabbit erythrocytes, various concentrations of the anti-Ba antibody were added to a fixed concentration of normal human serum (10%) and the assay was performed as described above. The data were recorded and analyzed with a SpectraMax plate reader and software.

As shown in FIG. 10, addition of serum in the absence of factor Ba antibody resulted in lysis of the cells and a dramatic reduction in light scattering. Addition of increasing concentrations of the antibody caused a decrement in erythrocyte lysis, with 125 ug/ml antibody completely blocking C5b-9-mediated cellular destruction. These results confirm that monoclonal antibodies that bind and block factor Ba interaction with properdin-bound C3b are potent reagents that can completely abrogate the effects of the complement pathway.

### Example 7

### Anti-Ba Prevents Complement and Cellular Activation in Whole Blood Model of Cardiopulmonary Bypass

To test the effect of an anti-factor Ba monoclonal antibody on inhibition of complement and cellular activation in cardiopulmonary bypass (CPB), a tubing loop model of CPB as described by Gong, J. R. et al. was utilized. Whole blood from a healthy donor was collected into a 7-ml vacutainer tube containing 5 U of heparin/mnl of blood. Polyvinyl chloride tubing like that used during CPB was filled with 2.0 ml of the heparinized human blood and closed into a loop with a short piece of silicon tubing and rotated vertically in a water bath for 2 hours at 37° C. After incubation, blood samples were transferred into 1.7 ml siliconized eppendorf tubes and analyzed for complement and cellular activation.

The plasma samples were diluted to 10% with sample diluent buffer and the amounts of C3a, C5b-9, elastase, and TNF-alpha were determined using ELISA assay kits following the manufacturer's instructions.

The blood aliquots following the tubing loop were stained with fluorescent antibodies to detect and quantify the activated neutrophils, monocytes and platelets. To stain neutrophils and monocytes, a 50 μl aliquot of blood was stained with neutrophil or monocyte specific antibodies. The aliquot of blood was stained with 20 μl of FITC-CD15 (neutrophils) or FITC-CD-14 (monocytes) antibody and 20 μl of PE-CD11b (both) antibody in 100 μl staining buffer (1% BSA in PBS). Following a twenty-minute incubation at room temperature, 2 ml of FACS lysing solution was be added and lysing continued for 20 minutes to allow complete lysing of red blood cells. The supernatant was aspirated and the cells were re-suspended in wash buffer (0.1% BSA in PBS). Cells of interest were identified in a forward-side scatter plot and then plotted in a FITC vs. side scatter plot to identify and separate the FITC positive population. The FITC positive populations of neutrophils or monocytes were then plotted in a dual quadrant plot with PE-CD11b label on the Y-axis and FITC-CD15 or FITC-CD14 on the X-axis. To determine the shift in the PE-CD 11b, PE data (x-axis) was plotted against the cell# (Y-axis) and compared against treated and untreated controls. Platelets were stained with 100 μl of staining buffer (1% BSA in PBS/azide) containing 20 μl of FITC labeled CD61 antibody and 20 μl of PE labeled CD62P antibody in 5 ml polypropylene tubes. Following 20-minute incubation at room temperature, 2 ml of FACS Lysing solution was added and the treated sample was allowed to incubate at room temperature for 20 minutes. Samples were centrifuged at 1200 g for 5 minutes. The supernatant was aspirated and the cells re-suspended in wash buffer (0.1% BSA in PBS/azide). The micro-centrifuge tubes are spun again, the supernatant aspirated, and the cells re-suspended in 0.5 ml of 0.1% para-formaldehyde. Platelets were analyzed for shift in CD62P staining.

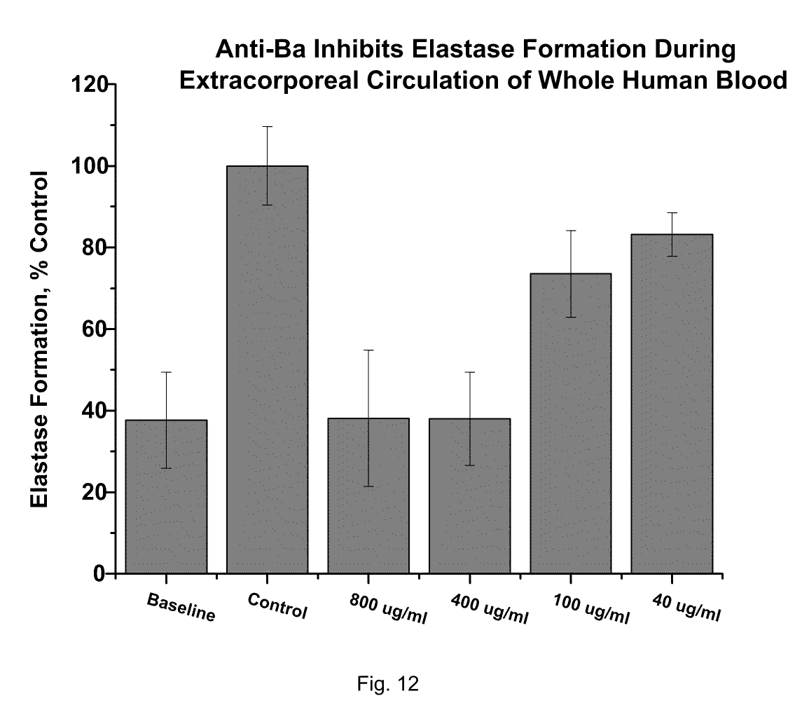

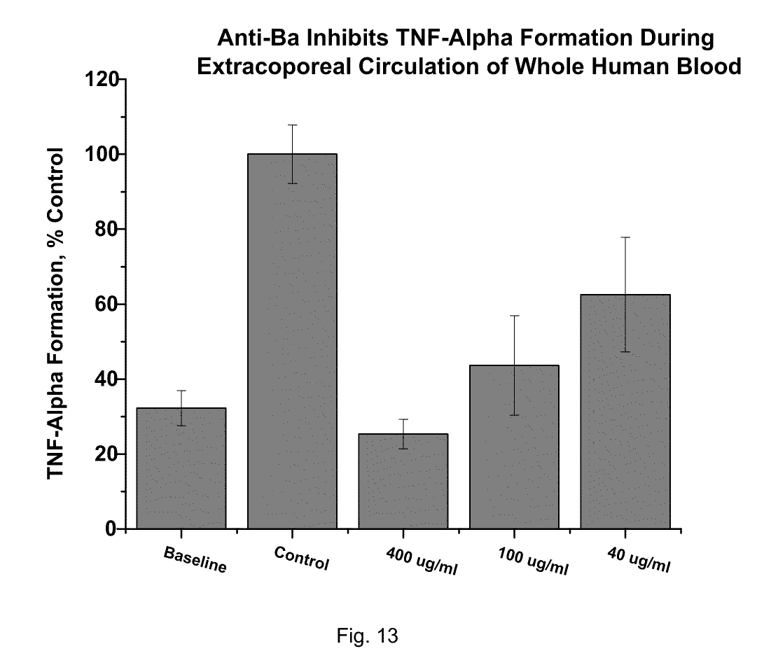

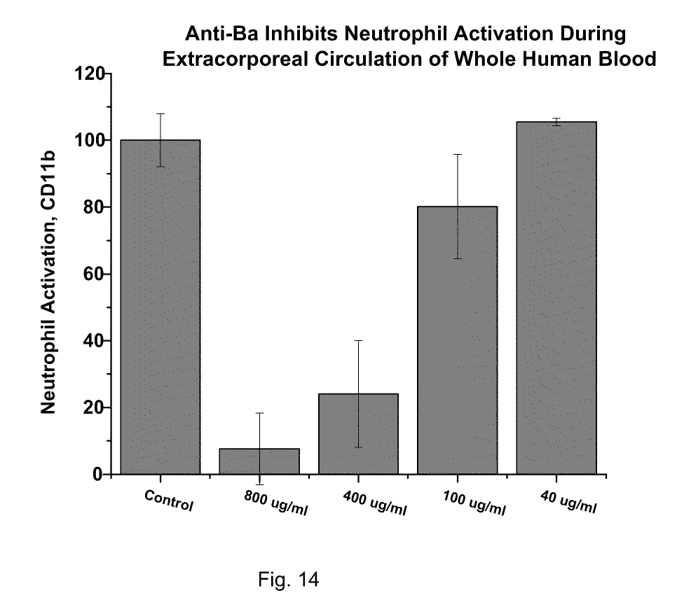

As shown in Anti-Ba inhibits C3a formation (FIG. 11), C5b-9 formation (FIG. 12), Elastase formation (FIG. 13), TNF-alpha (FIG. 14), Neutrophil activation (FIG. 15), Monocyte activation (FIG. 16), and platelet activation (FIG. 17) in whole blood model of cardiopulmonary bypass.

### INCORPORATION BY REFERENCE

All references cited herein, including patents, patent applications, papers, textbooks, and the like, and the references cited therein, to the extent that they are not already, are hereby incorporated herein by reference in their entirety. In addition, the following references are also incorporated by reference herein in their entirety, including the references cited in such references:

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

Having described the invention, the following is claimed:

1. A method of inhibiting factor Ba dependent complement activation in a subject, comprising administering to the subject an amount of an anti-factor Ba antibody or fragment thereof effective to inhibit factor Ba dependent alternative complement activation.

2. The method of claim 1 wherein the anti-factor Ba antibody or fragment thereof specifically binds to Factor Ba protein sequences involved in Factor B binding to C3b or factor B cleavage into Ba protein

3. The method of claim 1 wherein the anti-factor Ba antibody or fragment thereof prevents the formation of C3bBb.

4. The method of claim 1 wherein the anti-factor Ba antibody or fragment specifically binds to C3b binding sequences on Factor Ba.

5. The method of claim 1 wherein the anti-factor Ba antibody or fragment thereof prevents the release of Ba

6. The method of claim 1 wherein anti-factor Ba antibody or fragment thereof prevents only AP activation but not CP activation

7. The method of claim 1 wherein anti-factor Ba antibody or fragment thereof prevents activation of all three complement pathways.

8. The method of claim 1 wherein the anti-factor Ba antibody or fragment thereof prevents the formation of C3a, C5a, and C5b-9

9. The method of claim 1 wherein the anti-factor Ba antibody or fragment thereof prevents the formation on TNF alpha and neutrophil elastase

10. The method of claim 1 wherein the anti-factor Ba antibody or fragment thereof prevents the activation of neutrophils, monocytes and platelets

11. The method of claim 1 wherein the anti-factor Ba antibody or fragment thereof prevents the formation of leukocyte-platelet aggregates

12. The method of claim 11 wherein the antibody or fragment thereof is monoclonal.

13. The method of claim 11 wherein the antibody or fragment thereof is polyclonal.

14. The method of claim 11 wherein the antibody or fragment thereof is a recombinant antibody.

15. The method of claim 11 wherein the antibody has reduced effector function.

16. The method of claim 11 wherein the antibody is a chimeric, humanized or human antibody.

17. The method of claim 1 wherein the antibody is produced in a factor B deficient transgenic animal.

18. A method of treating a subject suffering from a Factor Ba-dependent complement mediated condition, comprising: administering to subject an amount of an anti-factor Ba antibody or fragment thereof effective to inhibit Factor Ba-dependent complement activation.

19. The method of claim 18, wherein the condition comprises at least one of a vascular condition, an ischemia-reperfusion injury, atherosclerosis, an inflammatory gastrointestinal disorder, a pulmonary condition, an extracorporeal reperfusion procedure, a musculoskeletal condition, a renal condition, a skin condition, an organ or tissue transplant procedure, a nervous system disorder, a blood disorder, a urogenital condition, diabetes, a neoplastic disorder, malignancy, endocrine disorder, or an ophthalmologic condition.

20. The method of claim 18 wherein the vascular condition comprises at least one of a cardiovascular condition, a cerebrovascular condition, a peripheral vascular condition, a renovascular condition, a mesenteric/enteric vascular condition, revascularization to transplants and/or replants, vasculitis, Henoch-Schonlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease, venous gas embolus (VGE), and restenosis following stent placement, rotational atherectomy or percutaneous transluminal coronary angioplasty (PTCA).

21. The method of claim 18, wherein the ischemia-reperfusion injury is associated with at least one of aortic aneurysm repair, cardiopulmonary bypass, vascular reanastomosis in connection with organ transplants and/or extremity/digit replantation, stroke, myocardial infarction, and hemodynamic resuscitation following shock and/or surgical procedures.

22. The method of claim 18, wherein the inflammatory gastrointestinal disorder is selected from the group consisting of pancreatitis, Crohn's disease, ulcerative colitis, irritable bowel syndrome and diverticulitis.

23. The method of claim 18 wherein the pulmonary condition is selected from the group consisting of acute respiratory distress syndrome, transfusion-related acute lung injury, ischemia/reperfusion acute lung injury, chronic obstructive pulmonary disease, asthma, Wegener's granulomatosis, antiglomerular basement membrane disease (Goodpasture's disease), meconium aspiration syndrome, bronchiolitis obliterans syndrome, idiopathic pulmonary fibrosis, acute lung injury secondary to bum, non-cardiogenic pulmonary edema, transfusion-related respiratory depression and emphysema.

24. The method of claim 18 wherein the extracorporeal reperfusion procedure is selected from the group consisting of hemodialysis, plasmapheresis, leukopheresis, extracorporeal membrane oxygenator (ECMO), heparin-induced extracorporeal membrane oxygenation LDL precipitation (HELP) and cardiopulmonary bypass (CPB).

25. The method of claim 18 wherein the musculoskeletal condition is selected from the group consisting of osteoarthritis, rheumatoid arthritis, juvenile rheumatoid arthritis, gout, neuropathic arthropathy, psoriatic arthritis, spondyloarthropathy, crystalline arthropathy and systemic lupus erythematosus (SLE).

26. The method of claim 18 wherein the renal condition is selected from the group consisting of mesangioproliferative glomerulonephritis, membranous glomerulonephritis, membranoproliferative glomerulonephritis (mesangiocapillary glomerulonephritis), acute postinfectious glomerulonephritis (poststreptococcal glomerulonephritis), cryoglobulinemic glomerulonephritis, lupus nephritis, Henoch-Schonlein purpura nephritis and IgA nephropathy.

27. The method of claim 18 wherein the skin condition is selected from the group consisting of psoriasis, autoimmune bullous dermatoses, eosinophilic spongiosis, bullous pemphigoid, epidermolysis bullosa acquisita, herpes gestationis, thermal bum injury and chemical burn injury.

28. The method of claim 18 wherein the transplant procedure is selected from the group consisting of organ allotransplantation, organ xenotransplantation organ and tissue graft.

29. The method of claim 18 wherein the nervous system disorder or injury is selected from the group consisting of multiple sclerosis, myasthenia gravis, Huntington's disease, amyotrophic lateral sclerosis, Guillain Barre syndrome, reperfusion following stroke, degenerative discs, cerebral trauma, Parkinson's disease, Alzheimer's disease, Miller-Fisher syndrome, cerebral trauma and/or hemorrhage, demyellination and meningitis.

30. The method of claim 18 wherein the blood disorder is selected from the group consisting of sepsis, severe sepsis, septic shock, acute respiratory distress syndrome resulting from sepsis, systemic inflammatory response syndrome, hemorrhagic shock, hemolytic anemia, autoimmune thrombotic thrombocytopenic purpura and hemolytic uremic syndrome.

31. The method of claim 18 wherein the urogenital condition is selected from the group consisting of painful bladder disease, sensory bladder disease, chronic abacterial cystitis, interstitial cystitis, infertility, placental dysfunction and miscarriage and pre-eclampsia.

32. The method of claim 18 wherein the diabetes comprises at least one of nonobese diabetes (Type-1 diabetes or Insulin-dependent diabetes mellitus) and/or complications associated with Type-1 or Type-2 (adult onset) diabetes.

33. The method of claim 18 wherein the complication associated with Type 1 or Type 2 diabetes is selected from the group consisting of angiopathy, neuropathy and retinopathy.

34. The method of claim 18 wherein the neoplastic condition includes a subject that has undergone, is undergoing, or will undergo chemotherapeutic treatment and/or radiation therapy.

35. The method of claim 18 wherein the endocrine disorder is selected from the group consisting of Hashimoto's thyroiditis, stress, anxiety, hormonal disorders involving regulated release of prolactin, growth or other insulin-like growth factor and adrenocorticotropin from the pituitary.

36. The method of claim 18 wherein the ophthalmologic condition is age-related macular degeneration.

37. A method of inhibiting factor Ba dependent complement activation in a subject, comprising administering to the subject an amount of an anti-factor Ba antibody or fragment thereof effective to inhibit factor Ba dependent classical, lectin and alternative complement activation via the amplification loop.

38. The method of claim 37 wherein the anti-factor Ba antibody or fragment thereof specifically binds to Factor Ba protein sequences involved in Factor B binding to C3b or factor Factor B cleavage into Ba protein.

39. The method of claim 37 wherein the anti-factor Ba antibody or fragment thereof prevents the formation of C3bBb.

40. The method of claim 37 wherein the anti-factor Ba antibody or fragment thereof prevents the release of Ba.

41. The method of claim 37 wherein the anti-factor Ba antibody or fragment thereof prevents the formation of C3a, C5a, and C5b-9

42. The method of claim 37 wherein the anti-factor Ba antibody or fragment thereof prevents the formation on TNF alpha and neutrophil elastase.

43. The method of claim 37 wherein the anti-factor Ba antibody or fragment thereof prevents the activation of neutrophils, monocytes and platelets

44. The method of claim 37 wherein the anti-factor Ba antibody or fragment thereof prevents the formation of leukocyte-platelet aggregates
